(19) United States
(12) Patent Application Publication
(10) Pub. No.: US 2014/0121409 A1
(43) Pub. Date: May 1, 2014

(54) Processes for the Production of Acrylic Acids and Acrylates Using Multiple Reactors

(71) Applicant: CELANESE INTERNATIONAL CORPORATION, Irving, TX (US)

(72) Inventors: Tianshu Pan, Houston, TX (US); Josefina T. Chapman, Houston, TX (US); Sean Mueller, Pasadena, TX (US)

(73) Assignee: CELANESE INTERNATIONAL CORPORATION, Irving, TX (US)

(21) Appl. No.: 13/664,856

(22) Filed: Oct. 31, 2012

(51) Int. Cl. C07C 51/347

(52) U.S. Cl. CPC C07C 51/347
USPC 562/599; 422/619

(57) ABSTRACT

In one embodiment, the present invention relates to a process for producing an acrylate product comprising the step of providing a reaction system comprising at least one reaction zone and at least one regeneration zone. Each reaction zone and regeneration zone has a size and comprises a catalyst. A ratio of the combined size of the at least one reaction zone to the combined size of the at least one regeneration zone ranges between 1:1 to 6:1. The process further comprises the step of reacting, in the at least one reaction zone, a reaction mixture comprising an alkanoic acid and an alkylenating agent to form a crude acrylate product. The process further comprises the step of regenerating, in the at least one regeneration zone, the respective catalyst. The process further comprises the step of separating at least a portion of the crude acrylate products to form a purified acrylate product.

### FIELD OF THE INVENTION

The present invention relates generally to the production of acrylic acid via the aldol condensation reaction of an alkanoic acid and an alkylenating agent. More specifically, the present invention relates to specific reactor configurations for use in the aldol condensation reaction.

### BACKGROUND OF THE INVENTION

α,β-unsaturated acids, particularly acrylic acid and methacrylic acid, and the ester derivatives thereof are useful organic compounds in the chemical industry. These acids and esters are known to readily polymerize or co-polymerize to form homopolymers or copolymers. Often the polymerized acids are useful in applications such as superabsorbents, dispersants, flocculants, and thickeners. The polymerized ester derivatives are used in coatings (including latex paints), textiles, adhesives, plastics, fibers, and synthetic resins.

Because acrylic acid and its esters have long been valued commercially, many methods of production have been developed. One exemplary acrylic acid ester production process utilizes: (1) the reaction of acetylene with water and carbon monoxide; and/or (2) the reaction of an alcohol and carbon monoxide, in the presence of an acid, e.g., hydrochloric acid, and nickel tetracarbonyl, to yield a crude product comprising the acrylate ester as well as hydrogen and nickel chloride. Another conventional process involves the reaction of ketene (often obtained by the pyrolysis of acetone or acetic acid) with formaldehyde, which yields a crude product comprising acrylic acid and either water (when acetic acid is used as a pyrolysis reactant) or methane (when acetone is used as a pyrolysis reactant). These processes have become obsolete for economic, environmental, or other reasons.

More recent acrylic acid production processes have relied on the gas phase oxidation of propylene, via acrolein, to form acrylic acid. The reaction can be carried out in single- or two-step processes but the latter is favored because of higher yields. The oxidation of propylene produces acrolein, acrylic acid, acetaldehyde and carbon oxides. Acrylic acid from the primary oxidation can be recovered while the acrolein is fed to a second step to yield the crude acrylic acid product, which comprises acrylic acid, water, small amounts of acetic acid, as well as impurities such as furfural, acrolein, and propionic acid. Purification of the crude product may be carried out by azeotropic distillation. Although this process may show some improvement over earlier processes, this process suffers from production and/or separation inefficiencies. In addition, this oxidation reaction is highly exothermic and, as such, creates an explosion risk. As a result, more expensive reactor design and metallurgy are required. Also, the cost of propylene is often prohibitive.

The aldol condensation reaction of formaldehyde and acetic acid and/or carboxylic acid esters has been disclosed in literature. This reaction forms acrylic acid and is often conducted over a catalyst. For example, condensation catalysts consisting of mixed oxides of vanadium and phosphorus were investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987).

US Patent Publication No. 2012/0071688 discloses a process for preparing acrylic acid from methanol and acetic acid in which the methanol is partially oxidized to formaldehyde in a heterogeneously catalyzed gas phase reaction. The product gas mixture thus obtained and an acetic acid source are used to obtain a reaction gas input mixture that comprises acetic acid and formaldehyde. The acetic acid is used in excess over the formaldehyde. The formaldehyde present in reaction gas input mixture is aldol-condensed with the acetic acid via heterogeneous catalysis to form acrylic acid. Unconverted acetic acid still present alongside the acrylic acid in the product gas mixture is removed therefrom and is recycled to the reaction gas input mixture. The acetic acid conversions in the disclosed reactions, however, may leave room for improvement.

Although the aldol condensation reaction is disclosed, there has been little if any disclosure relating to the effects multiple reaction zones on overall reaction system efficiency.

Thus, the need exists for a cost-effective process for producing purified acrylate product, e.g., acrylic acid, which provides suitable yield, catalyst performance, and/or separation efficiency.

The references mentioned above are hereby incorporated by reference.

### BRIEF DESCRIPTION OF DRAWINGS

The invention is described in detail below with reference to the appended drawings, wherein like numerals designate similar parts.

FIG. 1 is a schematic of an acrylic acid reaction/separation system in accordance with an embodiment of the present invention.

FIG. 2 is a schematic diagram of a reaction zone in accordance with one embodiment of the present invention.

FIG. 3 is a schematic diagram of a reaction zone in accordance with one embodiment of the present invention.

FIG. 4 is a schematic diagram of a reaction zone in accordance with one embodiment of the present invention.

FIG. 5 is a schematic of an acrylic acid reaction/separation system in accordance with an embodiment of the present invention.

FIG. 6 is a schematic of an acrylic acid reaction/separation system in accordance with an embodiment of the present invention.

FIG. 7 is a schematic of an acrylic acid reaction/separation system in accordance with an embodiment of the present invention.

### SUMMARY OF THE INVENTION

In one embodiment, the invention relates to a process for producing an acrylate product. The process comprising the steps of providing a reaction system comprising at least one reaction zone and at least one regeneration zone. Each reaction zone may have a size and may comprise a respective catalyst. Each regeneration zone may have a size and may comprise a respective catalyst. Preferably, a ratio of the combined size of the at least one reaction zone to the combined size of the at least one regeneration zone ranges from 1:1 to 6:1. The process further comprises the step of reacting, in the at least one reaction zone, a reaction mixture comprising an alkanoic acid and an alkylenating agent under conditions effective to form a crude acrylate product. The process may further comprise the step of regenerating, in the at least one regeneration zone, the respective catalyst. The process may further comprise the step of separating at least a portion of the crude acrylate products to form a purified acrylate product.

In one embodiment, the invention relates to a process for producing an acrylate product. The process comprises the step of providing a reaction system comprising three zones. Each of the three zones comprises, e.g., contains, a respective catalyst. For example, a first zone may comprise a first catalyst, a second zone may comprise a second catalyst, and a third zone may comprise a third catalyst. The process further comprises the step of reacting, in two of the three zones, a reaction mixture comprising an alkanoic acid and an alkylenating agent under conditions effective to form a crude acrylate product. The reaction may be conducted over the respective catalyst in each of the zones. The process further comprises the step of regenerating, in the other of the three zones, the respective catalyst. In one embodiment, the regenerating occurs in the reactors that are not utilized in the reacting step. The process may further comprise the step of separating at least a portion of the crude acrylate products, e.g., a portion of the combined crude acrylate products from each of the reactors, to form a purified acrylate product.

In another embodiment, the reaction system may comprise four zones. Each of the four zones comprises, e.g., contains, a respective catalyst. In this case, the reacting step may comprise the step of reacting, in three of the four zones, a reaction mixture comprising an alkanoic acid and an alkylenating agent under conditions effective to form a crude acrylate product. The process may further comprise the step of regenerating, in the other of the four zones, the respective catalyst. In one embodiment, the regenerating occurs in the reactors that are not utilized in the reacting step.

In another embodiment, the reaction system may comprise five zones. Each of the five zones comprises, e.g., contains, a respective catalyst. In this case, the reacting step may comprise the step of reacting, in four of the five zones, a reaction mixture comprising an alkanoic acid and an alkylenating agent under conditions effective to form a crude acrylate product. The process may further comprise the step of regenerating, in the other of the five zones, the respective catalyst. In one embodiment, the regenerating occurs in the reactors that are not utilized in the reacting step.

In another embodiment, the reaction system may comprise six zones. Each of the six zones comprises, e.g., contains, a respective catalyst. In this case, the reacting step may comprise the step of reacting, in five of the six zones, a reaction mixture comprising an alkanoic acid and an alkylenating agent under conditions effective to form a crude acrylate product. The process may further comprise the step of regenerating, in the other of the six zones, the respective catalyst. In one embodiment, the regenerating occurs in the reactors that are not utilized in the reacting step.

### DETAILED DESCRIPTION OF THE INVENTION

### Introduction

Production of unsaturated carboxylic acids such as acrylic acid and methacrylic acid and the ester derivatives thereof via most conventional processes have been limited by economic and environmental constraints. In the interest of finding a new reaction path, the aldol condensation reaction of acetic acid and an alkylenating agent, e.g., formaldehyde, has been investigated. This reaction may yield a unique crude product that comprises, inter alia, a higher amount of (residual) formaldehyde, which is generally known to add unpredictability and problems to separation schemes. Although the aldol condensation reaction of acetic acid and formaldehyde is known, there has been little if any disclosure relating to the effects of reactant feed parameters on the aldol condensation crude product.

In typical aldol condensation reaction systems, a single reaction zone, e.g., a single reactor, is employed. Although this reaction system may facilitate the aldol condensation reaction, the reaction efficiencies associated therewith leave much room for improvement. As one example, catalyst contained in the reaction zone may deactivate rapidly when a single reaction zone is utilized. As a result, catalyst must be frequently replaced and/or may require regeneration. This replacement and/or regeneration of catalyst may lead to process inefficiencies, e.g., loss of production time, high maintenance burdens, and increased equipment expenditures.

In one embodiment of the present invention, the aldol condensation reaction is conducted in a reaction system comprising at least one reaction zone and at least one regeneration zone. Each reaction zone and each regeneration zone comprise a catalyst. In preferred embodiments, each reaction zone may comprise a reactor, and each reactor may be charged with a respective catalyst. In one embodiment, the catalyst in each individual zone may vary from zone to zone. In one embodiment, the catalyst in each individual zone may be similar or the same. Each reaction zone has a size. The size of the reaction zone may be associated with the amount of catalyst that may be contained by the reaction zone. Likewise, each regeneration zone has a size. The size of the regeneration zone may be associated with the amount of catalyst that may be contained by the regeneration zone. In one embodiment, the sizes of the various individual zones may vary from zone to zone. In one embodiment, the sizes of the various individual zones may be similar or the same. In one embodiment, the sizes of the at least one reaction zone and/or the at least one regeneration zone range from 5 m³ to 100 m³, e.g., from 25 m³ to 75 m³, from 30 m³ to 70 m³, or from 40 m³ to 60 m³. In one embodiment, the sizes of the reaction zone(s) and/or the regeneration zones are greater than 5 m³, e.g., greater than 25 m³, greater than 30 m³ or greater than 40 m³. In one embodiment, the sizes of the reaction zone(s) and/or the regeneration zones are less than 100 m³, e.g., less than 75 m³, e.g., less than 70 m³ or less than 60 m³.

It has now been discovered that when a ratio of the combined size of the reaction zones to the combined size of the regeneration zones is maintained within a specific range, e.g., from 1:1 to 6:1, surprising and unexpected production efficiencies are demonstrated, as compared to other configurations, e.g., configurations utilizing other configurations of reaction zones and regeneration zones. For example: 1) the amount of catalyst necessary to maintain reaction efficiency and/or yield is low; 2) the number of reaction zones and regeneration zones is low; and/or 3) maintenance burdens associated with the reaction zones and regeneration zones are low. As a result of the combination of these unexpected improvements, overall reaction efficiencies and cost effectiveness are achieved, as compared to conventional reaction scheme configurations.

Without being bound by theory, it is believed that when the ratio of the combined size of the reaction zones to the combined size of the regeneration zones is maintained within the specific range of the present invention, a rate of catalyst regeneration in the regeneration zone(s) is similar to or the same as a rate of deactivation in the combined reaction zones. As a result, the efficiency of the catalyst replacement scheme is improved, the overall cost associated with the catalyst package is reduced, and production reliability and stability are increased. Generally speaking, the rate of catalyst regeneration per unit catalyst may not be the same as the rate of catalyst deactivation. When the ratios of the present invention are utilized, as one quantity of catalyst becomes sufficiently deactivated, another similar quantity of catalyst is regenerated. Thus, loss of catalyst down time is minimized. In contrast, in a conventional reaction system that employs a ratio outside of the inventive range, the rate of catalyst regeneration is much higher or much lower than the rate of deactivation and the catalyst replacement scheme is inefficient.

When the rate of catalyst regeneration is much higher than the rate of deactivation, the regeneration of the regeneration zone catalyst is complete before the reaction zone catalyst is deactivated. As a result, the catalyst in the regeneration zone is not used to catalyze reactants and, accordingly, does not yield acrylate product. On the other hand, when the rate of catalyst regeneration is much lower than the rate of deactivation, the catalyst in the reaction zone(s) is operating at a deactivated level while the regeneration is being performed. As a result, the efficiency of the reaction is low. Also, in cases where the ratio is high, the maintenance required to change out the many reaction zones with regenerated catalyst significantly reduces process efficiencies.

In embodiments where the sizes of the reaction zones and the regeneration zones are similar, it has now been discovered that when a single regeneration zone is utilized in combination with two, three, four or five reaction zones, the above-mentioned improvements in production efficiencies as well as the associated cost-related improvements are surprisingly and unexpectedly demonstrated. These improvements are based on comparisons with other configurations, e.g., configurations utilizing combinations of reaction zones and regeneration zones outside the scope of the present invention.

In one embodiment, the present invention relates to a process for producing acrylic acid, methacrylic acid, and/or the salts and esters thereof. As used herein, acrylic acid, methacrylic acid, and/or the salts and esters thereof, collectively or individually, may be referred to as “acrylate product” or “acrylate products.” The use of the terms acrylic acid, methacrylic acid, or the salts and esters thereof, individually, does not exclude the other acrylate products, and the use of the term acrylate product does not require the presence of acrylic acid, methacrylic acid, and the salts and esters thereof.

In one embodiment, the process comprises the step of providing a reaction system comprising at least one reaction zone and at least one regeneration zone, as discussed above. In one embodiment, the reaction system comprises at least two reaction zones, e.g., at least three reaction zones or at least four reaction zones. In such cases, the reaction zones may be configured in parallel. In one embodiment, the at least one reaction zone and/or the at least one regeneration zone are configured in parallel. The ratio of the combined size of the at least one reaction zone to the combined size of the at least one regeneration zone ranges from 1:1 to 6:1, e.g., from 1.1:1 to 5.9:1, from 1.5:1 to 5.5:1 or from 2:1 to 4:1. In one embodiment, the ratio of the combined size of the at least one reaction zone to the combined size of the at least one reaction zone is greater than 1:1, e.g., greater than 1.1:1, greater than 2:1, or greater than 3:1. In one embodiment, the ratio of the combined size of the at least one reaction zone to the combined size of the at least one reaction zone is less than 6:1, e.g., less than 5.9:1, less than 5:1, or less than 4:1. The at least one reaction zone may be fed with a reactant feed stream comprising reactants, e.g., alkanoic acid and alkylenating agent. In the reaction zones, the reactants may be reacted over the respective catalyst. As a result of the reaction, each of the at least one reaction zone yields a crude acrylate product. The at least one regeneration zone may be fed with a regeneration feed, e.g., oxygen, nitrogen, air, carbon dioxide, other inerts, or combinations thereof. The regeneration feed contacts the respective (deactivated) catalyst, thus yielding a regenerated catalyst. The deactivation may be a function of carbon deposits that form on the surface of the catalyst. The formation of the carbon is prohibitive to reaction. The contacting of the deactivated catalyst with the deactivating agent removes, e.g., burns, the carbon formations from the catalyst. Preferably, the number of reaction zones and regeneration zones, combined, ranges from 2 to 6, e.g., from 3 to 5. In one embodiment, the number of reaction zones and regeneration zones, combined, is less than 6, e.g., less than 5 or less than 4. In one embodiment, the number of reaction zones and regeneration zones, combined, is greater than 1, e.g., greater than 2 or greater than 3. In one embodiment, the reaction system comprises three reaction zones and one regeneration zone.

In one embodiment, the process further comprises the step of reacting, in the at least one reaction zone, a reaction mixture comprising an alkanoic acid and an alkylenating agent under conditions effective to form a crude acrylate product. The reaction mixtures may be fed to the at least one reaction zone via reactant feed stream(s), as discussed above.

The process, in one embodiment, further comprises the step of regenerating catalyst in the at least one regeneration zone. Preferably, the regenerating is conducted in the at least one regenerating zone and not in the at least one reaction zone. As mentioned above, the regeneration may be achieved by contacting the (deactivated) catalyst with a regeneration feed stream. The regeneration feed stream(s) may comprise at least one regeneration agent selected from the group consisting of oxygen, nitrogen, air, carbon dioxide, other inerts, and combinations thereof. When contacted with the regeneration feed, impurities that have formed on the deactivated catalyst, e.g., on the catalyst sites, may be removed, e.g., may be burned off.

The process may further comprise the step of separating at least a portion of the crude acrylate products, e.g., the crude acrylate products that exit the at least one reaction zone, to form a purified acrylate product.

In some embodiments, the sizes of the reaction zones and the regeneration zones are similar. For example, the reaction zones and the regeneration zones may all comprise similar reactors. In one such embodiment, the process comprises the step of providing a reaction system comprising three zones. As discussed above, each zone comprises a catalyst. The process further comprises the step of reacting, in two of the three zones, e.g., in the two reaction zones, a reaction mixture comprising an alkanoic acid and an alkylenating agent under conditions effective to form a crude acrylate product. The reaction mixtures may be fed to the two of the three zones via reactant feed streams, as discussed above. The zones that are fed with the reactant feed stream(s) may be considered reaction zones. Preferably, the reaction system comprises two reaction zones (and one regeneration zone). In one embodiment, the reaction system comprises no more than three zones. In one embodiment, the process further comprises the step of regenerating catalyst. The regenerating may be conducted in the other of the three zones. The regeneration may be achieved by contacting the (deactivated) catalyst of the other of the three zones with a regeneration feed stream, as discussed above. The zone(s) that are fed with the regeneration feed stream(s) may be considered regeneration zones.

In one embodiment, the size of the regeneration zone is not greater than the size of the first reaction zone, the second reaction zone, or the third reaction zone. In one embodiment, the size of the regeneration zone is not greater than an average size of the reaction zones.

In one embodiment, the process comprises the step of providing a reaction system comprising four zones. As discussed above, each zone comprises a catalyst. The process further comprises the step of reacting, in three of the four zones, e.g., in the three reaction zones, a reaction mixture comprising an alkanoic acid and an alkylenating agent under conditions effective to form a crude acrylate product. The reaction mixtures may be fed to the three of the four zones via reactant feed streams, as discussed above. The zones that are fed with the reactant feed stream(s) may be considered reaction zones. Preferably, the reaction system comprises three reaction zones (and one regeneration zone). In one embodiment, the reaction system comprises no more than four zones. In one embodiment, the process further comprises the step of regenerating catalyst. The regenerating may be conducted in the other of the four zones. The regeneration may be achieved by contacting the (deactivated) catalyst of the other of the four zones with a regeneration feed stream, as discussed above. The zone(s) that are fed with the regeneration feed stream(s) may be considered regeneration zones.

In one embodiment, the process comprises the step of providing a reaction system comprising five zones. As discussed above, each zone comprises a catalyst. The process further comprises the step of reacting, in four of the five zones, e.g., in the four reaction zones, a reaction mixture comprising an alkanoic acid and an alkylenating agent under conditions effective to form a crude acrylate product. The reaction mixtures may be fed to the four of the five zones via reactant feed streams, as discussed above. The zones that are fed with the reactant feed stream(s) may be considered reaction zones. Preferably, the reaction system comprises four reaction zones (and one regeneration zone). In one embodiment, the reaction system comprises no more than five zones. In one embodiment, the process further comprises the step of regenerating catalyst. The regenerating may be conducted in the other of the five zones. The regeneration may be achieved by contacting the (deactivated) catalyst of the other of the five zones with a regeneration feed stream, as discussed above. The zone(s) that are fed with the regeneration feed stream(s) may be considered regeneration zones.

In one embodiment, the process comprises the step of providing a reaction system comprising six zones. As discussed above, each zone comprises a catalyst. The process further comprises the step of reacting, in five of the six zones, e.g., in the five reaction zones, a reaction mixture comprising an alkanoic acid and an alkylenating agent under conditions effective to form a crude acrylate product. The reaction mixtures may be fed to the five of the six zones via reactant feed streams, as discussed above. The zones that are fed with the reactant feed stream(s) may be considered reaction zones. Preferably, the reaction system comprises five reaction zones (and one regeneration zone). In one embodiment, the reaction system comprises no more than six zones. In one embodiment, the process further comprises the step of regenerating catalyst. The regenerating may be conducted in the other of the six zones. The regeneration may be achieved by contacting the (deactivated) catalyst of the other of the six zones with a regeneration feed stream, as discussed above. The zone(s) that are fed with the regeneration feed stream(s) may be considered regeneration zones.

In some embodiments, the inventive process may change reaction zones to regeneration zones, and vice versa. As noted above, the reaction zones are fed with reactant feeds and the regeneration zones are fed with regeneration feeds. By changing the feed of a particular zone, the function of the zone may be changed. For example, in some embodiments, as the catalyst in a reaction zone becomes deactivated, the deactivated reaction zone may require regeneration. The regeneration may be achieved by changing the feed stream of the reaction zone from a reactant feed to a regeneration feed, thus changing the reaction zone to a regeneration zone. As another example, as the catalyst in a regeneration zone becomes regenerated, the regenerated zone may be available for use as a reaction zone to produce acrylate product. Thus, in one embodiment, the feed stream of the initial regeneration zone may be changed from a regeneration feed stream to a reactant feed stream, thus changing the regeneration zone to a reaction zone. It has now been discovered that, by changing from reaction zone(s) to regeneration zone(s) in accordance with embodiments of the present invention, the process advantageously achieves high catalyst efficiency. As such, reaction efficiencies and/or yields can be maintained using smaller amounts of catalyst and/or smaller reactors, which, beneficially, reduces overall system cost.

In one embodiment, the point at which the reaction zones are changed is dependent upon the activity of the catalyst contained therein. In one embodiment, the process of the present invention comprises the step of monitoring catalyst activity of the catalyst(s). Catalyst activity may be monitored in at least one, e.g., at least two, or at least three, of the reaction zones. In one embodiment, the activity of the catalyst in each of the reaction zones is monitored. The manner in which the catalyst activity is monitored may vary widely. For example, catalyst activity may be based on reactant conversion, e.g., acetic acid conversion, on reactant selectivity, e.g., acetic acid selectivity to acrylic acid, and/or on yield. In one embodiment, catalyst activity may be based on acrylate product yield, e.g., acrylic acid yield.

In one embodiment, when catalyst activity of a reaction zone is determined to be below a desired level, the reaction in the reaction zone may be discontinued. In one embodiment, the discontinued reaction zone may be regenerated. In one embodiment, the process comprises the step of discontinuing the reacting in at least one of the at least one reaction zone. The decision to discontinue the reacting may be based on the monitored catalyst activity. For example, the reacting may be discontinued when catalyst activity in a particular reaction zone is less than 95% of the desired level, e.g., less than 90%, less than 85%, or less than 75%. In one embodiment, the reacting may be discontinued when acetic acid conversion in a particular reaction zone is less than 50%, e.g., less than 40%, less than 30% less than 20% or less than 10%. In one embodiment, the reacting may be discontinued when selectivity to acrylate product is less than 95%, e.g., less than 90%, less than 85%, or less than 75%. In one embodiment, the reacting may be discontinued when acrylate product yield in a particular reaction zone is less than 30%, e.g., less than 25% or less than 20%.

In one embodiment, when the reaction in a particular reaction zone is discontinued, the discontinued reaction zone may be regenerated. The process may further comprise the step of regenerating the catalyst in the discontinued reaction zone(s). In this case, the discontinued reaction zone is changed to a regeneration zone. As discussed above, this change may be achieved by changing the feed stream that is fed to the particular reaction zone, e.g., by changing the reactant feed stream to a regeneration feed stream.

In one embodiment, the regenerating in the at least one regeneration zone may be discontinued. As such, the process may further comprise the step of discontinuing the regenerating in at least one of the at least one regeneration zone. In one embodiment, the decision to discontinue may be based on the monitored catalyst activity of the catalyst in at least one of the reaction zones. In one embodiment, the decision to discontinue may be based on the regeneration level of the catalyst in the at least one regeneration zone. In some embodiments, when the regenerating is discontinued in a regenerating zone, the process may further comprise the step of reacting, in the at least one discontinued regeneration zone, a reaction mixture comprising an alkanoic acid and an alkylenating agent over the respective catalyst and under conditions effective to form a crude acrylate product. In this case, the discontinued regeneration zone is changed to a reaction zone. As discussed above, this change may be achieved by changing the feed stream that is fed to the particular regeneration zone, e.g., by changing the regeneration feed stream to a reactant feed stream.

### Crude Acrylate Product

The aldol condensation reaction of the present invention, unlike most conventional acrylic acid-containing crude products, yields crude acrylate products (from each reaction zone) comprising acrylate product and a significant portion of at least one alkylenating agent. Preferably, the at least one alkylenating agent is formaldehyde. For example, the crude product streams may comprise at least 0.5 wt % alkylenating agent(s), e.g., at least 1 wt %, at least 5 wt %, at least 7 wt %, at least 10 wt %, or at least 25 wt %. In terms of ranges, the crude product streams may comprise from 0.5 wt % to 50 wt % alkylenating agent(s), e.g., from 1 wt % to 45 wt %, from 1 wt % to 25 wt %, from 1 wt % to 10 wt %, or from 5 wt % to 10 wt %. In terms of upper limits, the crude product streams may comprise at most 50 wt % alkylenating agent(s), e.g., at most 45 wt %, at most 25 wt %, or at most 10 wt %.

The crude acrylate products comprise at least 1 wt % acrylate product, e.g., at least 5 wt % or at least 10 wt %. In terms of ranges, the crude product streams may comprise from 1 wt % to 75 wt % acrylate product, e.g., from 1 wt % to 50 wt %, from 5 wt % to 50 wt %, or from 10 wt % to 40 wt %. In terms of upper limits, the crude product streams may comprise at most 75 wt % alkylenating agent(s), e.g., at most 50 wt %, or at most 40 wt %. Preferably the acrylate product is acrylic acid.

In one embodiment, the crude product streams of the present invention further comprise water. For example, the crude product streams may comprise less than 50 wt % water, e.g., less than 40 wt %, less than 30 wt %, or less than 25 wt %. In terms of ranges, the crude product streams may comprise from 1 wt % to 50 wt % water, e.g., from 5 wt % to 40 wt %, from 10 wt % to 30 wt %, or from 15 wt % to 25 wt %. In terms of lower limits, the crude product streams may comprise at least 1 wt % water, e.g., at least 5 wt %, at least 10 wt %, or at least 15 wt %.

In one embodiment, the crude product streams of the present invention comprise very little, if any, of the impurities found in most conventional acrylic acid crude product streams. For example, the crude product streams of the present invention may comprise less than 1000 wppm of such impurities (either as individual components or collectively), e.g., less than 500 wppm, less than 100 wppm, less than 50 wppm, or less than 10 wppm. Exemplary impurities include acetylene, ketene, beta-propiolactone, higher alcohols, e.g., C₂₊, C₃₊, or C₄₊, and combinations thereof. Importantly, the crude product streams of the present invention comprise very little, if any, furfural and/or acrolein. In one embodiment, the crude product streams comprise substantially no furfural and/or acrolein, e.g., no furfural and/or acrolein. In one embodiment, the crude product streams comprise less than less than 500 wppm acrolein, e.g., less than 100 wppm, less than 50 wppm, or less than 10 wppm. In one embodiment, the crude product streams comprise less than less than 500 wppm furfural, e.g., less than 100 wppm, less than 50 wppm, or less than 10 wppm. Furfural and acrolein are known to act as detrimental chain terminators in acrylic acid polymerization reactions. Also, furfural and/or acrolein are known to have adverse effects on the color of purified product and/or to subsequent polymerized products.

In addition to the acrylic acid and the alkylenating agent, the crude product streams may further comprise acetic acid, water, propionic acid, and light ends such as oxygen, nitrogen, carbon monoxide, carbon dioxide, methanol, methyl acetate, methyl acrylate, acetaldehyde, hydrogen, and acetone. In one embodiment, because the exemplary oxygen amounts are employed, carbon monoxide and/or carbon dioxide production in inhibited. For example, the crude acrylate product may comprise less than 20 wt % carbon monoxide and/or carbon dioxide, e.g., less than 15 wt %, less than 10 wt % or less than 5 wt %. In one embodiment, a weight ratio of carbon monoxide and carbon dioxide, combined, to acrylate product is less than 0.50, e.g., less than 0.46, less than 0.4, or less than 0.25.

Exemplary compositional data for the crude product streams are shown in Table 1. The compositional data in Table 1 reflects the composition of the crude product stream that is fed to the separation zone if 1) nitrogen dilution conditions are not employed; or 2) nitrogen dilution conditions are employed and at least a major portion, preferably substantially all, of the nitrogen used in the nitrogen dilution is removed from the crude product stream before being fed to the separation zone. Components other than those listed in Table 1 may also be present in the crude product streams.

TABLE 1
CRUDE ACRYLATE PRODUCT COMPOSITIONS

| | Conc. | Conc. | Conc. | Conc. |
|---|---|---|---|---|
| Component | (wt %) | (wt %) | (wt %) | (wt %) |
| Acrylic Acid | 1 to 75 | 1 to 50 | 5 to 50 | 10 to 40 |
| Alkylenating Agent(s) | 0.5 to 50 | 1 to 45 | 1 to 25 | 1 to 10 |
| Acetic Acid | 1 to 90 | 1 to 70 | 5 to 50 | 10 to 50 |
| Water | 1 to 50 | 5 to 40 | 10 to 30 | 15 to 25 |
| Propionic Acid | 0.01 to 10 | 0.1 to 10 | 0.1 to 5 | 0.1 to 1 |
| Oxygen | 0.01 to 10 | 0.1 to 10 | 0.1 to 5 | 0.1 to 1 |
| Nitrogen | 0.1 to 20 | 0.1 to 10 | 0.5 to 5 | 0.5 to 4 |
| Carbon Monoxide | 0.01 to 10 | 0.1 to 10 | 0.1 to 5 | 0.5 to 3 |
| Carbon Dioxide | 0.01 to 10 | 0.1 to 10 | 0.1 to 5 | 0.5 to 3 |
| Other Light Ends | 0.01 to 10 | 0.1 to 10 | 0.1 to 5 | 0.5 to 3 |

In one embodiment, the crude acrylate products exiting the reaction zones may be combined with one another and directed to a separation zone. In one embodiment the crude acrylate products are directed to the separation zone individually. In one embodiment, some crude acrylate products may be combined with others and directed to the separation zone, while other crude acrylate products are individually directed to the separation zone.

Any suitable reaction and/or separation scheme may be employed to form the crude product stream as long as the reaction provides the crude product stream components that are discussed above. For example, in some embodiments, the acrylate product stream is formed by contacting an alkanoic acid, e.g., acetic acid, or an ester thereof with an alkylenating agent, e.g., a methylenating agent, for example formaldehyde, under conditions effective to form the crude acrylate product. Preferably, the contacting is performed over a suitable catalyst. The crude product stream may be the reaction product of the alkanoic acid-alkylenating agent reaction. In a preferred embodiment, the crude product stream is the reaction product of the aldol condensation reaction of acetic acid and formaldehyde, which is conducted over a catalyst comprising vanadium and titanium. In one embodiment, the crude product stream is the product of a reaction where methanol and acetic acid are combined to generate formaldehyde in situ. The aldol condensation then follows. In one embodiment, a methanol-formaldehyde solution is reacted with acetic acid to form the crude product stream.

The alkanoic acid, or an ester of the alkanoic acid, may be of the formula R′—CH₂—COOR, where R and R′ are each, independently, hydrogen or a saturated or unsaturated alkyl or aryl group. As an example, R and R′ may be a lower alkyl group containing for example 1-4 carbon atoms. In one embodiment, an alkanoic acid anhydride may be used as the source of the alkanoic acid. In one embodiment, the reaction is conducted in the presence of an alcohol, preferably the alcohol that corresponds to the desired ester, e.g., methanol. In addition to reactions used in the production of acrylic acid, the inventive catalyst, in other embodiments, may be employed to catalyze other reactions.

The alkanoic acid, e.g., acetic acid, may be derived from any suitable source including natural gas, petroleum, coal, biomass, and so forth. As examples, acetic acid may be produced via methanol carbonylation, acetaldehyde oxidation, ethylene oxidation, oxidative fermentation, and anaerobic fermentation.

As petroleum and natural gas prices fluctuate, becoming either more or less expensive, methods for producing acetic acid and intermediates such as methanol and carbon monoxide from alternate carbon sources have drawn increasing interest. In particular, when petroleum is relatively expensive compared to natural gas, it may become advantageous to produce acetic acid from synthesis gas (“syngas”) that is derived from any available carbon source. U.S. Pat. No. 6,632,752, which is hereby incorporated by reference, for example, teaches a method of retrofitting a methanol plant for the manufacture of acetic acid. By retrofitting a methanol plant, the large capital costs associated with carbon monoxide generation for a new acetic acid plant are significantly reduced or largely eliminated. All or part of the syngas is diverted from the methanol synthesis loop and supplied to a separator unit to recover carbon monoxide and hydrogen, which are then used to produce acetic acid.

In some embodiments, at least some of the raw materials for the above-described aldol condensation process may be derived partially or entirely from syngas. For example, the acetic acid may be formed from methanol and carbon monoxide, both of which may be derived from syngas. For example, the methanol may be formed by steam reforming syngas, and the carbon monoxide may be separated from syngas. In other embodiments, the methanol may be formed in a carbon monoxide unit, e.g., as described in EP2076480; EP1927380; EP2072490; EP1914219; EP1904426; EP2072487; EO2072492; EP2072486; EP2060553; EP1741692; EP1907744; EP2060555; EP2186787; EP2072488; and U.S. Pat. No. 7,842,844, which are hereby incorporated by reference. Of course, this listing of methanol sources is merely exemplary and is not meant to be limiting. In addition, the above-identified methanol sources, inter alia, may be used to form the formaldehyde, e.g., in situ, which, in turn may be reacted with the acetic acid to form the acrylic acid. The syngas, in turn, may be derived from variety of carbon sources. The carbon source, for example, may be selected from the group consisting of natural gas, oil, petroleum, coal, biomass, and combinations thereof. Syngas or hydrogen may also be obtained from bio-derived methane gas, such as bio-derived methane gas produced by landfills or agricultural waste.

In another embodiment, in addition to the acetic acid formed via methanol carbonylation, some additional acetic acid may be formed from the fermentation of biomass and may be used in the hydrogenation step. The fermentation process preferably utilizes an acetogenic process or a homoacetogenic microorganism to ferment sugars to acetic acid producing little, if any, carbon dioxide as a by-product. The carbon efficiency for the fermentation process preferably is greater than 70%, greater than 80% or greater than 90% as compared to conventional yeast processing, which typically has a carbon efficiency of about 67%. Optionally, the microorganism employed in the fermentation process is of a genus selected from the group consisting of Clostridium, Lactobacillus, Moorella, Thermoanaerobacter, Propionibacterium, Propionispera, Anaerobiospirillum, and Bacteriodes, and in particular, species selected from the group consisting of Clostridium formicoaceticum, Clostridium butyricum, Moorella thermoacetica, Thermoanaerobacter kivui, Lactobacillus delbrukii, Propionibacterium acidipropionici, Propionispera arboris, Anaerobiospirillum succinicproducens, Bacteriodes amylophilus and Bacteriodes ruminicola. Optionally in this process, all or a portion of the unfermented residue from the biomass, e.g., lignans, may be gasified to form hydrogen that may be used in the hydrogenation step of the present invention. Exemplary fermentation processes for forming acetic acid are disclosed in U.S. Pat. Nos. 6,509,180; 6,927,048; 7,074,603; 7,507,562; 7,351,559; 7,601,865; 7,682,812; and 7,888,082, the entireties of which are incorporated herein by reference. See also U.S. Pub. Nos. 2008/0193989 and 2009/0281754, the entireties of which are incorporated herein by reference.

Examples of biomass include, but are not limited to, agricultural wastes, forest products, grasses, and other cellulosic material, timber harvesting residues, softwood chips, hardwood chips, tree branches, tree stumps, leaves, bark, sawdust, off-spec paper pulp, corn, corn stover, wheat straw, rice straw, sugarcane bagasse, switchgrass, miscanthus, animal manure, municipal garbage, municipal sewage, commercial waste, grape pumice, almond shells, pecan shells, coconut shells, coffee grounds, grass pellets, hay pellets, wood pellets, cardboard, paper, plastic, and cloth. See, e.g., U.S. Pat. No. 7,884,253, the entirety of which is incorporated herein by reference. Another biomass source is black liquor, a thick, dark liquid that is a byproduct of the Kraft process for transforming wood into pulp, which is then dried to make paper. Black liquor is an aqueous solution of lignin residues, hemicellulose, and inorganic chemicals.

Methanol carbonylation processes suitable for production of acetic acid are described in U.S. Pat. Nos. 7,208,624, 7,115,772, 7,005,541, 6,657,078, 6,627,770, 6,143,930, 5,599,976, 5,144,068, 5,026,908, 5,001,259, and 4,994,608, all of which are hereby incorporated by reference.

U.S. Pat. No. RE 35,377, which is hereby incorporated by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form syn gas. The syn gas is converted to methanol which may be carbonylated to acetic acid. U.S. Pat. No. 5,821,111, which discloses a process for converting waste biomass through gasification into syn gas, as well as U.S. Pat. No. 6,685,754 are hereby incorporated by reference.

In one optional embodiment, the acetic acid that is utilized in the condensation reaction comprises acetic acid and may also comprise other carboxylic acids, e.g., propionic acid, esters, and anhydrides, as well as acetaldehyde and acetone. In one embodiment, the acetic acid fed to the condensation reaction comprises propionic acid. For example, the acetic acid fed to the reaction may comprise from 0.001 wt % to 15 wt % propionic acid, e.g., from 0.001 wt % to 0.11 wt %, from 0.125 wt % to 12.5 wt %, from 1.25 wt % to 11.25 wt %, or from 3.75 wt % to 8.75 wt %. Thus, the acetic acid feed stream may be a cruder acetic acid feed stream, e.g., a less-refined acetic acid feed stream.

As used herein, “alkylenating agent” means an aldehyde or precursor to an aldehyde suitable for reacting with the alkanoic acid, e.g., acetic acid, to form an unsaturated acid, e.g., acrylic acid, or an alkyl acrylate. In preferred embodiments, the alkylenating agent comprises a methylenating agent such as formaldehyde, which preferably is capable of adding a methylene group (═CH₂) to the organic acid. Other alkylenating agents may include, for example, acetaldehyde, propanal, butanal, aryl aldehydes, benzyl aldehydes, alcohols, and combinations thereof. This listing is not exclusive and is not meant to limit the scope of the invention. In one embodiment, an alcohol may serve as a source of the alkylenating agent. For example, the alcohol may be reacted in situ to form the alkylenating agent, e.g., the aldehyde.

The alkylenating agent, e.g., formaldehyde, may be derived from any suitable source. Exemplary sources may include, for example, aqueous formaldehyde solutions, anhydrous formaldehyde derived from a formaldehyde drying procedure, trioxane, diether of methylene glycol, and paraformaldehyde. In a preferred embodiment, the formaldehyde is produced via a methanol oxidation process, which reacts methanol and oxygen to yield the formaldehyde.

In other embodiments, the alkylenating agent is a compound that is a source of formaldehyde. Where forms of formaldehyde that are not as freely or weakly complexed are used, the formaldehyde will form in situ in the condensation reactor or in a separate reactor prior to the condensation reactor. Thus for example, trioxane may be decomposed over an inert material or in an empty tube at temperatures over 750° C. or over an acid catalyst at over 100° C. to form the formaldehyde.

In one embodiment, the alkylenating agent corresponds to Formula I.

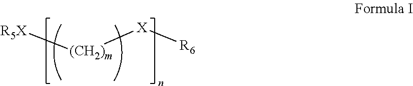

In Formula I, R₅ and R₆ may be independently selected from C₁-C₁₂ hydrocarbons, preferably, C₁-C₁₂ alkyl, alkenyl or aryl, or hydrogen. Preferably, R₅ and R₆ are independently C₁-C₆ alkyl or hydrogen, with methyl and/or hydrogen being most preferred. X may be either oxygen or sulfur, preferably oxygen; and n is an integer from 1 to 10, preferably 1 to 3. In some embodiments, m is 1 or 2, preferably 1.

In one embodiment, the compound of formula I may be the product of an equilibrium reaction between formaldehyde and methanol in the presence of water. In such a case, the compound of formula I may be a suitable formaldehyde source. In one embodiment, the formaldehyde source includes any equilibrium composition. Examples of formaldehyde sources include but are not restricted to methylal (1,1 dimethoxymethane); polyoxymethylenes —(CH₂—O)ᵢ— wherein i is from 1 to 100; formalin; and other equilibrium compositions such as a mixture of formaldehyde, methanol, and methyl propionate. In one embodiment, the source of formaldehyde is selected from the group consisting of 1,1 dimethoxymethane; higher formals of formaldehyde and methanol; and CH₃—O—(CH₂—O)ᵢ—CH₃ where i is 2.

The alkylenating agent may be used with or without an organic or inorganic solvent.

As discussed above, in some embodiments, the alkylenating agent that is reacted with the alkanoic acid may be provided to the process in the form of a crude alkylenating agent stream. The crude alkylenating agent stream comprises alkylenating agent, e.g., formaldehyde, and at least one other impurity, e.g., water and/or methanol. Preferably, the crude alkylenating agent stream comprises formalin. The term “formalin,” refers to a mixture of formaldehyde, methanol, and water. In one embodiment, formalin comprises from 37 wt % to 55 wt % formaldehyde, from 44 wt % to 60 wt % water, and from 0.01 wt % to 25 wt % methanol. In cases where a mixture of formaldehyde, methanol, and methyl propionate is used, the mixture comprises less than 10 wt % water, e.g., less than 5 wt % or less than 1 wt %. In accordance with the present invention, the crude alkylenating agent may be dehydrated to reduce impurity content in the crude alkylenating agent stream, e.g., to remove water from the crude alkylenating agent stream.

In some embodiments, the condensation reaction may achieve favorable conversion of acetic acid and favorable selectivity and productivity to acrylates. For purposes of the present invention, the term “conversion” refers to the amount of acetic acid in the feed that is converted to a compound other than acetic acid. Conversion is expressed as a percentage based on acetic acid in the feed. The conversion of acetic acid may be at least 10%, e.g., at least 20%, at least 40%, or at least 50%.

Selectivity, as it refers to the formation of acrylate product, is expressed as the ratio of the amount of carbon in the desired product(s) and the amount of carbon in the total products. This ratio may be multiplied by 100 to arrive at the selectivity. Preferably, the catalyst selectivity to acrylate products, e.g., acrylic acid and methyl acrylate, is at least 40 mol %, e.g., at least 50 mol %, at least 60 mol %, or at least 70 mol %. In some embodiments, the selectivity to acrylic acid is at least 30 mol %, e.g., at least 40 mol %, or at least 50 mol %; and/or the selectivity to methyl acrylate is at least 10 mol %, e.g., at least 15 mol %, or at least 20 mol %.

The terms “productivity” or “space time yield” as used herein, refers to the grams of a specified product, e.g., acrylate products, formed per hour during the condensation based on the liters of catalyst used. A productivity of at least 20 grams of acrylate product per liter catalyst per hour, e.g., at least 100 grams of acrylates per liters catalyst per hour or at least 200 grams of acrylates per liter catalyst per hour, is preferred. In terms of ranges, the productivity preferably is from 20 to 1000 grams of acrylates per liter catalyst per hour, e.g., from 20 to 700 grams of acrylates per liter catalyst per hour or from 100 to 600 grams of acrylates per liter catalyst per hour or from 200 to 600 grams of acrylates per liter catalyst per hour or from 70 to 700 grams of acrylates per liter catalyst per hour.

In one embodiment, the inventive process yields at least 1,800 kg/hr of finished acrylic acid, e.g., at least 3,500 kg/hr, at least 18,000 kg/hr, or at least 37,000 kg/hr.

Formation of alkanes, e.g., ethane, may be low, and ideally less than 2%, less than 1%, or less than 0.5% of the acetic acid passed over the catalyst is converted to alkanes, which have little value other than as fuel.

The alkanoic acid or ester thereof and alkylenating agent may be fed independently or after prior mixing to a reactor system, as described above. The reaction zone(s) of the present invention may comprise any suitable reactor or combination of reactors. Preferably, each reaction zone comprises one or more fixed bed reactors. In one embodiment, each reaction zone comprises one or more packed bed reactors. In one embodiment, the reactors utilized in the reaction zones are fixed bed reactors, e.g., shell-and-tube type fixed bed reactors. Of course, other suitable reactors such as a continuous stirred tank reactor or a fluidized bed reactor, may be employed.

In some embodiments, the alkanoic acid, e.g., acetic acid, and the alkylenating agent, e.g., formaldehyde, are fed to the respective reactor at a molar ratio of at least 0.10:1, e.g., at least 0.75:1 or at least 1:1. In terms of ranges the molar ratio of alkanoic acid to alkylenating agent may range from 0.10:1 to 10:1 or from 0.75:1 to 5:1. In some embodiments, the reaction of the alkanoic acid and the alkylenating agent is conducted with a stoichiometric excess of alkanoic acid. In these instances, acrylate selectivity may be improved. As an example the acrylate selectivity may be at least 10% higher than a selectivity achieved when the reaction is conducted with an excess of alkylenating agent, e.g., at least 20% higher or at least 30% higher. In other embodiments, the reaction of the alkanoic acid and the alkylenating agent is conducted with a stoichiometric excess of alkylenating agent.

The condensation reaction may be conducted at a temperature of at least 250° C., e.g., at least 70° C., or at least 750° C. In terms of ranges, the reaction temperature may range from 200° C. to 500° C., e.g., from 250° C. to 400° C., or from 250° C. to 750° C. Residence time in the reactor may range from 1 second to 200 seconds, e.g., from 1 second to 100 seconds. Reaction pressure is not particularly limited, and the reaction is typically performed near atmospheric pressure. In one embodiment, the reaction may be conducted at a pressure ranging from 0 kPa to 4100 kPa, e.g., from 3 kPa to 345 kPa, or from 6 kPa to 103 kPa. The acetic acid conversion, in some embodiments, may vary depending upon the reaction temperature.

In one embodiment, the reaction is conducted at a gas hourly space velocity (“GHSV”) greater than 600 hr⁻¹, e.g., greater than 1000 hr⁻¹ or greater than 2000 hr⁻¹. In one embodiment, the GHSV ranges from 600 hr⁻¹ to 10000 hr⁻¹, e.g., from 1000 hr⁻¹ to 8000 hr⁻¹ or from 1500 hr⁻¹ to 7500 hr⁻¹. As one particular example, when GHSV is at least 2000 hr⁻¹, the acrylate product STY may be at least 150 g/hr/liter.

In one embodiment, the unreacted components such as the alkanoic acid and formaldehyde as well as the inert or reactive gases that remain are recycled after sufficient separation from the desired product.

When the desired product is an unsaturated ester made by reacting an ester of an alkanoic acid ester with formaldehyde, the alcohol corresponding to the ester may also be fed to the respective reaction zone either with or separately from the other components. For example, when methyl acrylate is desired, methanol may be fed to the reactor. The alcohol, amongst other effects, reduces the quantity of acids leaving the reactor. It is not necessary that the alcohol is added at the beginning of the reactor and it may for instance be added in the middle or near the back, in order to effect the conversion of acids such as propionic acid, methacrylic acid to their respective esters without depressing catalyst activity. In one embodiment, the alcohol may be added downstream of the reactor.

### Catalyst Composition

The catalyst may be any suitable catalyst composition. As one example, condensation catalyst consisting of mixed oxides of vanadium and phosphorus have been investigated and described in M. Ai, J. Catal., 107, 201 (1987); M. Ai, J. Catal., 124, 293 (1990); M. Ai, Appl. Catal., 36, 221 (1988); and M. Ai, Shokubai, 29, 522 (1987). Other examples include binary vanadium-titanium phosphates, vanadium-silica-phosphates, and alkali metal-promoted silicas, e.g., cesium- or potassium-promoted silicas.

In a preferred embodiment, the inventive process employs a catalyst composition comprising vanadium, titanium, and optionally at least one oxide additive. The oxide additive(s), if present, are preferably present in the active phase of the catalyst. In one embodiment, the oxide additive(s) are selected from the group consisting of silica, alumina, zirconia, and mixtures thereof or any other metal oxide other than metal oxides of titanium or vanadium. Preferably, the molar ratio of oxide additive to titanium in the active phase of the catalyst composition is greater than 0.05:1, e.g., greater than 0.1:1, greater than 0.5:1, or greater than 1:1. In terms of ranges, the molar ratio of oxide additive to titanium in the inventive catalyst may range from 0.05:1 to 20:1, e.g., from 0.1:1 to 10:1, or from 1:1 to 10:1. In these embodiments, the catalyst comprises titanium, vanadium, and one or more oxide additives and have relatively high molar ratios of oxide additive to titanium.

In another embodiment, the inventive process employs a catalyst comprising vanadium, titanium, bismuth, tungsten, or mixtures thereof. In some embodiments, the catalyst comprises bismuth. In other embodiments, the catalyst comprises tungsten. Exemplary catalyst compositions include vanadium/titanium/bismuth, vanadium/titanium/tungsten, bismuth/tungsten, and vanadium/bismuth/tungsten.

In other embodiments, the catalyst may further comprise other compounds or elements (metals and/or non-metals). For example, the catalyst may further comprise phosphorus and/or oxygen. In these cases, the catalyst may comprise from 15 wt % to 45 wt % phosphorus, e.g., from 20 wt % to 35 wt % or from 23 wt % to 27 wt %; and/or from 30 wt % to 75 wt % oxygen, e.g., from 35 wt % to 65 wt % or from 48 wt % to 51 wt %.

In some embodiments, the catalyst further comprises additional metals and/or oxide additives. These additional metals and/or oxide additives may function as promoters. If present, the additional metals and/or oxide additives may be selected from the group consisting of copper, molybdenum, tungsten, nickel, niobium, and combinations thereof. Other exemplary promoters that may be included in the catalyst of the invention include lithium, sodium, magnesium, aluminum, chromium, manganese, iron, cobalt, calcium, yttrium, ruthenium, silver, tin, barium, lanthanum, the rare earth metals, hafnium, tantalum, rhenium, thorium, bismuth, antimony, germanium, zirconium, uranium, cesium, zinc, and silicon and mixtures thereof. Other modifiers include boron, gallium, arsenic, sulfur, halides, Lewis acids such as BF₃, ZnBr₂, and SnCl₄. Exemplary processes for incorporating promoters into catalyst are described in U.S. Pat. No. 5,764,824, the entirety of which is incorporated herein by reference.

If the catalyst comprises additional metal(s) and/or metal oxides(s), the catalyst optionally may comprise additional metals and/or metal oxides in an amount from 0.001 wt % to 30 wt %, e.g., from 0.01 wt % to 5 wt % or from 0.1 wt % to 5 wt %. If present, the promoters may enable the catalyst to have a weight/weight space time yield of at least 25 grams of acrylic acid/gram catalyst-h, e.g., least 50 grams of acrylic acid/gram catalyst-h, or at least 100 grams of acrylic acid/gram catalyst-h.

In some embodiments, the catalyst is unsupported. In these cases, the catalyst may comprise a homogeneous mixture or a heterogeneous mixture as described above. In one embodiment, the homogeneous mixture is the product of an intimate mixture of vanadium and titanium oxides, hydroxides, and phosphates resulting from preparative methods such as controlled hydrolysis of metal alkoxides or metal complexes. In other embodiments, the heterogeneous mixture is the product of a physical mixture of the vanadium and titanium phosphates. These mixtures may include formulations prepared from phosphorylating a physical mixture of preformed hydrous metal oxides. In other cases, the mixture(s) may include a mixture of preformed vanadium pyrophosphate and titanium pyrophosphate powders.

In another embodiment, the catalyst is a supported catalyst comprising a catalyst support in addition to the vanadium, titanium, oxide additive, and optionally phosphorous and oxygen, in the amounts indicated above (wherein the molar ranges indicated are without regard to the moles of catalyst support, including any vanadium, titanium, oxide additive, phosphorous or oxygen contained in the catalyst support). The total weight of the support (or modified support), based on the total weight of the catalyst, preferably is from 75 wt % to 99.9 wt %, e.g., from 78 wt % to 97 wt %, or from 80 wt % to 95 wt %. The support may vary widely. In one embodiment, the support material is selected from the group consisting of silica, alumina, zirconia, titania, aluminosilicates, zeolitic materials, mixed metal oxides (including but not limited to binary oxides such as SiO₂—Al₂O₃, SiO₂—TiO₂, SiO₂—ZnO, SiO₂—MgO, SiO₂—ZrO₂, Al₂O₃—MgO, Al₂O₃—TiO₂, Al₂O₃—ZnO, TiO₂—MgO, TiO₂—ZrO₂, TiO₂—ZnO, TiO₂—SnO₂) and mixtures thereof, with silica being one preferred support. In embodiments where the catalyst comprises a titania support, the titania support may comprise a major or minor amount of rutile and/or anatase titanium dioxide. Other suitable support materials may include, for example, stable metal oxide-based supports or ceramic-based supports. Preferred supports include silicaceous supports, such as silica, silica/alumina, a Group IIA silicate such as calcium metasilicate, pyrogenic silica, high purity silica, silicon carbide, sheet silicates or clay minerals such as montmorillonite, beidellite, saponite, pillared clays, other microporous and mesoporous materials, and mixtures thereof. Other supports may include, but are not limited to, iron oxide, magnesia, steatite, magnesium oxide, carbon, graphite, high surface area graphitized carbon, activated carbons, and mixtures thereof. These listings of supports are merely exemplary and are not meant to limit the scope of the present invention.

In some embodiments, a zeolitic support is employed. For example, the zeolitic support may be selected from the group consisting of montmorillonite, NH₄ ferrierite, H-mordenite-PVOx, vermiculite-1, H-ZSM5, NaY, H-SDUSY, Y zeolite with high SAR, activated bentonite, H-USY, MONT-2, HY, mordenite SAR 20, SAPO-34, Aluminosilicate (X), VUSY, Aluminosilicate (CaX), Re-Y, and mixtures thereof. H-SDUSY, VUSY, and H-USY are modified Y zeolites belonging to the faujasite family. In one embodiment, the support is a zeolite that does not contain any metal oxide modifier(s). In some embodiments, the catalyst composition comprises a zeolitic support and the active phase comprises a metal selected from the group consisting of vanadium, aluminum, nickel, molybdenum, cobalt, iron, tungsten, zinc, copper, titanium cesium bismuth, sodium, calcium, chromium, cadmium, zirconium, and mixtures thereof. In some of these embodiments, the active phase may also comprise hydrogen, oxygen, and/or phosphorus.

In other embodiments, in addition to the active phase and a support, the inventive catalyst may further comprise a support modifier. A modified support, in one embodiment, relates to a support that includes a support material and a support modifier, which, for example, may adjust the chemical or physical properties of the support material such as the acidity or basicity of the support material. In embodiments that use a modified support, the support modifier is present in an amount from 0.1 wt % to 50 wt %, e.g., from 0.2 wt % to 25 wt %, from 0.5 wt % to 15 wt %, or from 1 wt % to 8 wt %, based on the total weight of the catalyst composition.

In one embodiment, the support modifier is an acidic support modifier. In some embodiments, the catalyst support is modified with an acidic support modifier. The support modifier similarly may be an acidic modifier that has a low volatility or little volatility. The acidic modifiers may be selected from the group consisting of oxides of Group IVB metals, oxides of Group VB metals, oxides of Group VIB metals, iron oxides, aluminum oxides, and mixtures thereof. In one embodiment, the acidic modifier may be selected from the group consisting of WO₃, MoO₃, Fe₂O₃, Cr₂O₃, V₂O₅, MnO₂, CuO, Co₂O₃, Bi₂O₃, TiO₂, ZrO₂, Nb₂O₅, Ta₂O₅, Al₂O₃, B₂O₃, P₂O₅, and Sb₂O₃.

In another embodiment, the support modifier is a basic support modifier. The presence of chemical species such as alkali and alkaline earth metals, are normally considered basic and may conventionally be considered detrimental to catalyst performance. The presence of these species, however, surprisingly and unexpectedly, may be beneficial to the catalyst performance. In some embodiments, these species may act as catalyst promoters or a necessary part of the acidic catalyst structure such in layered or sheet silicates such as montmorillonite. Without being bound by theory, it is postulated that these cations create a strong dipole with species that create acidity.

Additional modifiers that may be included in the catalyst include, for example, boron, aluminum, magnesium, zirconium, and hafnium.

As will be appreciated by those of ordinary skill in the art, the support materials, if included in the catalyst of the present invention, preferably are selected such that the catalyst system is suitably active, selective and robust under the process conditions employed for the formation of the desired product, e.g., acrylic acid or alkyl acrylate. Also, the active metals and/or pyrophosphates that are included in the catalyst of the invention may be dispersed throughout the support, coated on the outer surface of the support (egg shell) or decorated on the surface of the support. In some embodiments, in the case of macro- and meso-porous materials, the active sites may be anchored or applied to the surfaces of the pores that are distributed throughout the particle and hence are surface sites available to the reactants but are distributed throughout the support particle.

The inventive catalyst may further comprise other additives, examples of which may include: molding assistants for enhancing moldability; reinforcements for enhancing the strength of the catalyst; pore-forming or pore modification agents for formation of appropriate pores in the catalyst, and binders. Examples of these other additives include stearic acid, graphite, starch, cellulose, silica, alumina, glass fibers, silicon carbide, and silicon nitride. Preferably, these additives do not have detrimental effects on the catalytic performances, e.g., conversion and/or activity. These various additives may be added in such an amount that the physical strength of the catalyst does not readily deteriorate to such an extent that it becomes impossible to use the catalyst practically as an industrial catalyst.

### Separation

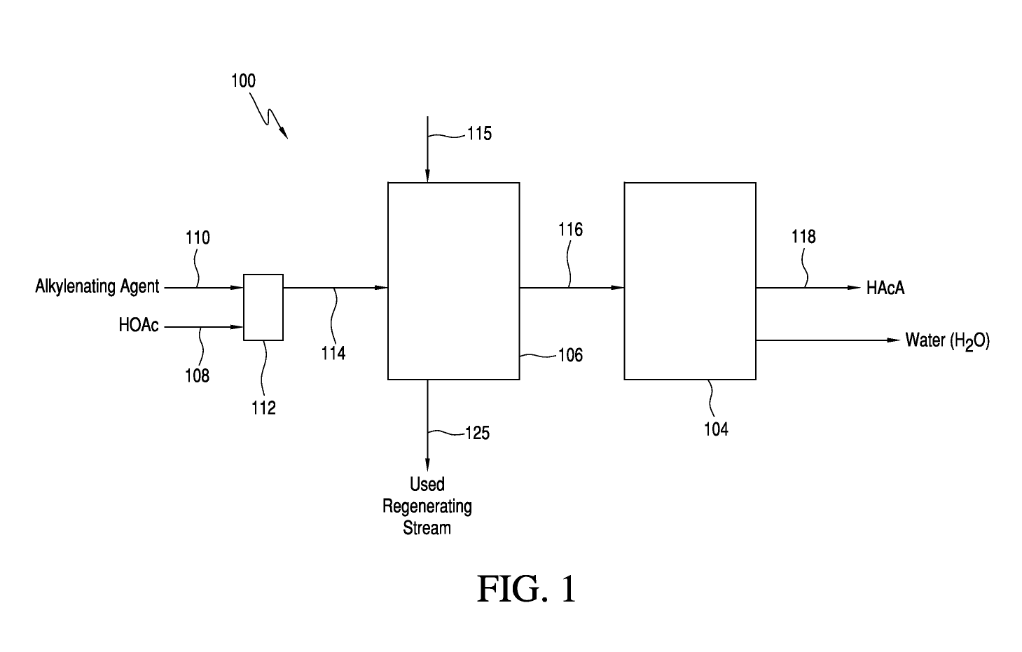

The unique crude acrylate product of the present invention may be separated in a separation zone to form a final purified product, e.g., a final acrylic acid product. FIG. 1 is a flow diagram depicting the formation of the crude acrylate product and the separation thereof to obtain an acrylate product 118. Acrylate product production system 100 comprises reaction scheme 102 and separation scheme 104. Reaction scheme 102 comprises reaction system 106, alkanoic acid feed, e.g., acetic acid feed, 108, alkylenating agent feed, e.g., formaldehyde feed 110, and vaporizer 112.

Acetic acid and formaldehyde are fed to vaporizer 112 via lines 108 and 110, respectively, to create a vapor feed stream, which exits vaporizer 112 via line 114 and is directed to reaction system 106. Optionally oxygen and/or methanol feeds, not shown, are fed to vaporizer 112. In other embodiments, not shown, any or all of the components of the reaction mixture, e.g., acetic acid, formaldehyde, oxygen, and/or methanol, may be fed directly to the reactor (not shown). In one embodiment, lines 108 and 110 (and optionally the oxygen and/or methanol feeds) may be combined and jointly fed to the vaporizer 112. The temperature of the vapor feed stream in line 114 is preferably from 200° C. to 600° C., e.g., from 250° C. to 500° C. or from 340° C. to 425° C.

Any feed that is not vaporized may be removed from vaporizer 112 and may be recycled or discarded. In one embodiment, for steady-state operation, all feeds are vaporized and used in the aldol condensation reaction. Further modifications and additional components to reaction scheme 102 and separation scheme 104 are described below.

Reaction system 106 is shown generally in FIG. 1. As discussed above, reaction system 106 comprises at least one reaction zone and at least one regeneration zone. The sizes and configuration of the reaction zones and regeneration zones of reaction system 106 are as described herein. Each of these zones comprises a catalyst. The aldol condensation reaction is conducted in reaction system 106 and crude acrylate product exits reaction system 106 via line 116. Regeneration stream 115 is fed to reaction system 106 and discharged via 125. Exemplary composition ranges for crude acrylate product 116 are shown in Table 1 above.

In one embodiment, one or more guard beds (not shown) may be used upstream of the reactor to protect the catalyst from poisons or undesirable impurities contained in the feed or return/purge streams. Such guard beds may be employed in the vapor or liquid streams. Suitable guard bed materials may include, for example, carbon, silica, alumina, ceramic, or resins. In one aspect, the guard bed media is functionalized, e.g., silver functionalized, to trap particular species such as sulfur or halogens.

The crude acrylate product in line 116 may be fed to separation scheme 104. Separation scheme 104 may comprise one or more separation units, e.g., two or more or three or more. Separation scheme 104 separates the crude acrylate product to yield a purified acrylate product stream, which exits via line 118.

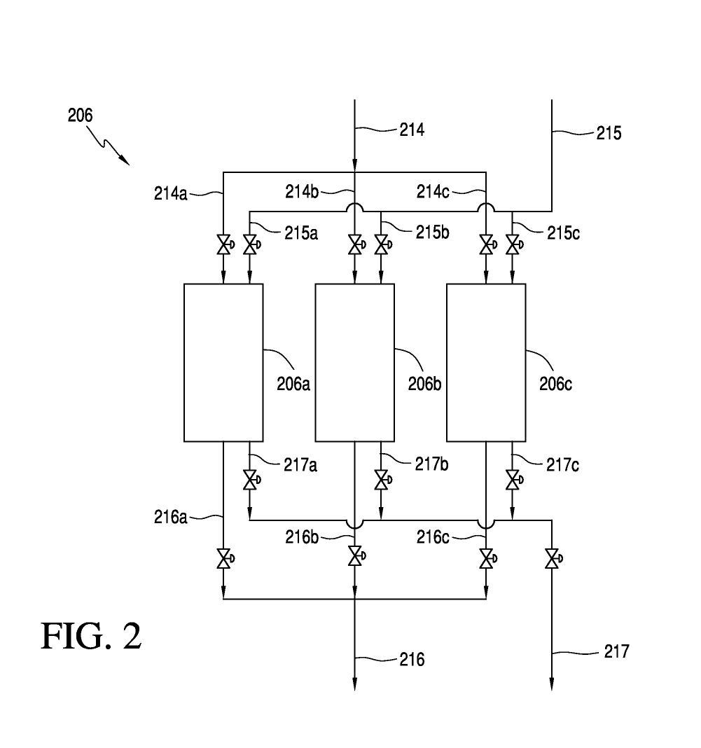

FIG. 2 shows exemplary reaction system 206 in accordance with one embodiment of the present invention. Reactant feed stream 214 is split into reactant feed streams 214a, 214b, and 214c, which may provide reactants to zones 206a, 206b, and 206c, respectively. Regeneration feed stream 215 is split into reactant feed streams 215a, 215b, and 215c, which may provide regeneration agent(s) to zones 206a, 206b, and 206c, respectively.

The reaction and regeneration are conducted in reaction system 206 as discussed herein. For example, at least one of zones 206a, 206b, and 206c is fed with the respective reactant feed stream and at least one of zones 206a, 206b, and 206c is fed with the respective regeneration feed stream. Preferably two of zones 206a, 206b, and 206c are fed with the respective reactant fed stream and one of zones 206a, 206b, and 206c is fed with the regeneration feed stream. In one embodiment, when a particular zone is fed with a reaction feed, that zone is not fed concurrently with a regeneration feed, and vice versa. A zone that is fed with a reaction feed may be referred to as a reaction zone and a zone that is fed with a regeneration feed may be referred to as a regeneration zone.

At least one of zones 206a, 206b, and 206c functions as a reaction zone. Each of the reaction zones yields a crude acrylate product, which exits the reaction zone via the respective exit line. Zone 206a comprises exit line 216a; zone 206b comprises exit line 216b; and zone 206c comprises exit line 216c.

At least one of zones 206a, 206b, and 206c functions as a regeneration zone. Each regeneration zone yields a used regeneration stream, which exits the regeneration zone via the respective exit line. Zone 206a comprises exit line 217a; zone 206b comprises exit line 217b; and zone 206c comprises exit line 217c.

When any of zones 206a, 206b, and/or 206c function as a reaction zone, the respective exit lines 216a, 216b, and/or 216c convey crude acrylate product to combined acrylate product stream 216. When any of zones 206a, 206b, and/or 206c function as a regeneration zone, little or no crude acrylate product exits the respective regeneration zone. When any of zones 206a, 206b, and/or 206c function as a regeneration zone, the respective exit lines 217a, 217b and/or 217c convey used regeneration agent to combined used regeneration stream 217, which may be recycled and re-used. When any of zones 206a, 206b, and/or 206c function as a reaction zone, no used regeneration agent exits the respective reaction zone.

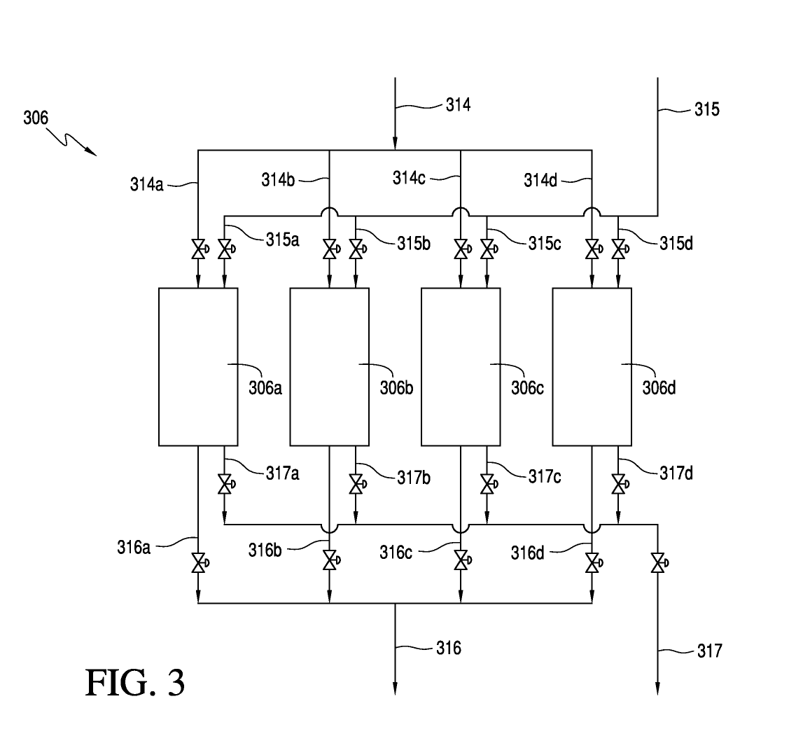

FIG. 3 shows exemplary reaction system 306 in accordance with the present invention. Reactant feed stream 314 is split into reactant feed streams 314a, 314b, 314c, and 314d, which may provide reactants to zones 306a, 306b, 306c, and 306d, respectively. Regeneration feed stream 315 is split into reactant feed streams 315a, 315b, 315c, and 315d, which may provide regeneration agent(s) to zones 306a, 306b, 306c, and 306d, respectively.

The reaction and regeneration are conducted in reaction system 306 as discussed herein. For example, at least one of zones 306a, 306b, 306c, and 306d, is fed with the respective reactant fed stream and at least one of zones 306a, 306b, 306c, and 306d, is fed with the respective regeneration feed stream. Preferably three of zones 306a, 306b, 306c, and 306d, are fed with the respective reactant fed stream and one of zones 306a, 306b, 306c, and 306d, is fed with the regeneration feed stream. In one embodiment, when a particular zone is fed with a reaction feed, that zone is not fed concurrently with a regeneration feed and vice versa. A zone that is fed with a reaction feed may be referred to as a reaction zone and a zone that is fed with a regeneration feed may be referred to as a regeneration zone.

At least one of zones 306a, 306b, 306c, and 306d, functions as a reaction zone. Each of the reaction zones yields a crude acrylate product, which exits the reaction zone via the respective exit line. Zone 306a comprises exit line 316a; zone 306b comprises exit line 316b; zone 306c comprises exit line 316c, and zone 306d comprises exit line 316d.

At least one of zones 306a, 306b, 306c, and 306d, functions as a regeneration zone. Each regeneration zone yields a used regeneration stream, which exits the regeneration zone via the respective exit line. Zone 306a comprises exit line 317a; zone 306b comprises exit line 317b; zone 306c comprises exit line 317c, and zone 306d comprises exit line 317d.

When any of zones 306a, 306b, 306c, and/or 306d function as a reaction zone, the respective exit lines 316a, 316b, 316c, and/or 316d convey crude acrylate product to combined acrylate product stream 316. When any of zones 306a, 306b, 306c, and/or 306d function as a regeneration zone, little or no crude acrylate product exits the respective regeneration zone. When any of zones 306a, 306b, 306c, and/or 306d function as a regeneration zone, the respective exit lines 317a, 317b, 317c, and/or 317c convey used regeneration agent to combined used regeneration stream 317, which may be recycled and re-used. When any of zones 306a, 306b, 306c, and/or 306d function as a reaction zone, no used regeneration agent exits the respective reaction zone.

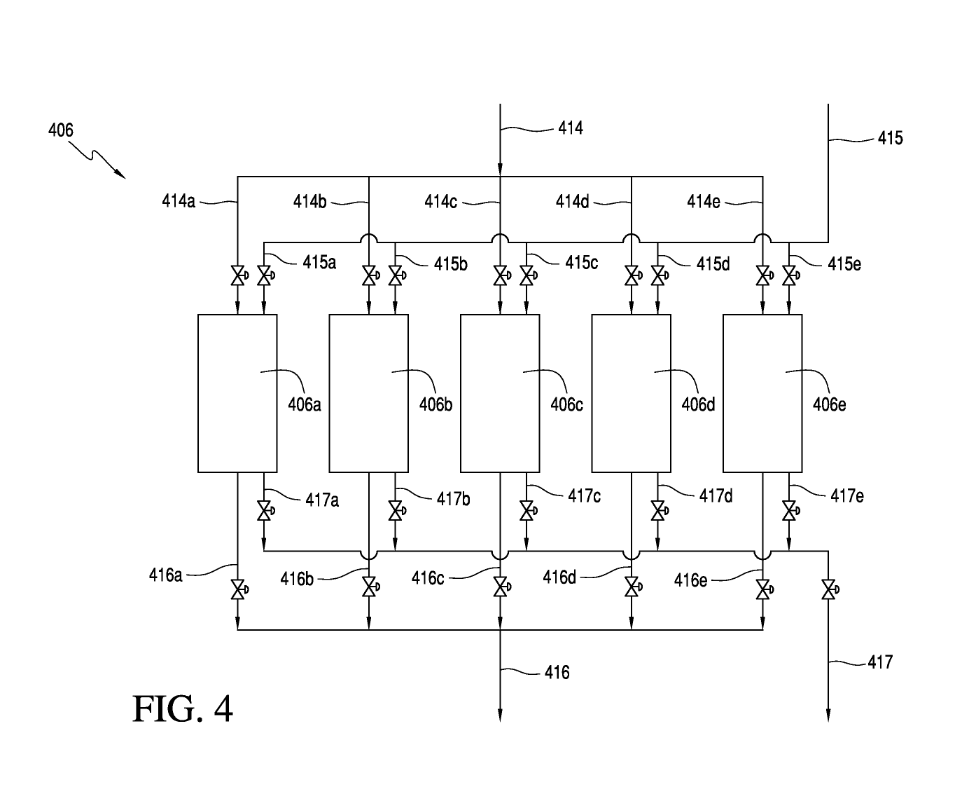

FIG. 4 shows exemplary reaction system 406 in accordance with the present invention. Reactant feed stream 414 is split into reactant feed streams 414a, 414b, 414c, 414d, and 414e, which may provide reactants to zones 406a, 406b, 406c, 406d, and 406e, respectively. Regeneration feed stream 415 is split into reactant feed streams 415a, 415b, 415c, 415d, and 415e, which may provide regeneration agent(s) to zones 406a, 406b, 406c, 406d, and 406e, respectively.

The reaction and regeneration are conducted in reaction system 406 as discussed herein. For example, at least one of zones 406a, 406b, 406c, 406d, and 406e is fed with the respective reactant feed stream and at least one of zones 406a, 406b, 406c, 406d, and 406e is fed with the respective regeneration feed stream. Preferably four of zones 406a, 406b, 406c, 406d, and 406e are fed with the respective reactant fed stream and one of zones 406a, 406b, 406c, 406d, and 406e is fed with the regeneration feed stream. In one embodiment, when a particular zone is fed with a reaction feed, that zone is not fed concurrently with a regeneration feed and vice versa. A zone that is fed with a reaction feed may be referred to as a reaction zone and a zone that is fed with a regeneration feed may be referred to as a regeneration zone.

At least one of zones 406a, 406b, 406c, 406d, and 406e functions as a reaction zone. Each of the reaction zones yields a crude acrylate product, which exits the reaction zone via the respective exit line. Zone 406a comprises exit line 416a; zone 406b comprises exit line 416b; zone 406c comprises exit line 416c, zone 406d comprises exit line 416d, and zone 406e comprises exit line 416e.

At least one of zones 406a, 406b, 406c, 406d, and 406e functions as a regeneration zone. Each regeneration zone yields a used regeneration stream, which exits the regeneration zone via the respective exit line. Zone 406a comprises exit line 417a; zone 406b comprises exit line 417b; zone 406c comprises exit line 417c, zone 406d comprises exit line 417d, and zone 406e comprises exit line 417e.

When any of zones 406a, 406b, 406c, 406d, and/or 406e function as a reaction zone, the respective exit lines 416a, 416b, 416c, 416d, and/or 416e convey crude acrylate product to combined acrylate product stream 416. When any of zones 406a, 406b, 406c, 406d, and/or 406e function as a regeneration zone, little or no crude acrylate product exits the respective regeneration zone. When any of zones 406a, 406b, 406c, 406d, and/or 406e function as a regeneration zone, the respective exit lines 417a, 417b, 417c, 417d and/or 417e convey used regeneration agent to combined used regeneration stream 417, which may be recycled and re-used. When any of zones 406a, 406b, 406c, 406d, and/or 406e function as a reaction zone, no used regeneration agent exits the respective reaction zone.

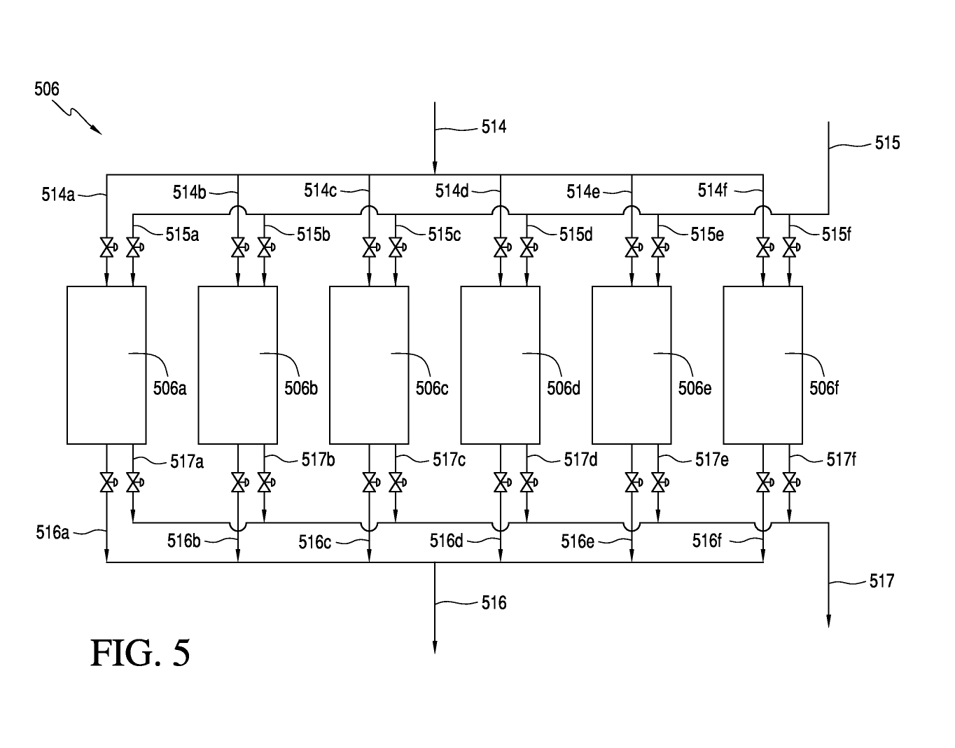

FIG. 5 shows exemplary reaction system 506 in accordance with the present invention. Reactant feed stream 514 is split into reactant feed streams 514a, 514b, 514c, 514d, 514e, and 514f, which may provide reactants to zones 506a, 506b, 506c, 506d, 506e, and 506f, respectively. Regeneration feed stream 515 is split into reactant feed streams 515a, 515b, 515c, 515d, 515e, and 515f, which may provide regeneration agent(s) to zones 506a, 506b, 506c, 506d, 506e, and 506f, respectively.

The reaction and regeneration are conducted in reaction system 506 as discussed herein. For example, at least one of zones 506a, 506b, 506c, 506d, 506e, and 506f is fed with the respective reactant feed stream and at least one of zones 506a, 506b, 506c, 506d, 506e, and 506f is fed with the respective regeneration feed stream. Preferably five of zones 506a, 506b, 506c, 506d, 506e, and 506f are fed with the respective reactant feed stream and one of zones 506a, 506b, 506c, 506d, 506e, and 506f is fed with the regeneration feed stream. In one embodiment, when a particular zone is fed with a reaction feed, that zone is not fed concurrently with a regeneration feed and vice versa. A zone that is fed with a reaction feed may be referred to as a reaction zone and a zone that is fed with a regeneration feed may be referred to as a regeneration zone.

At least one of zones 506a, 506b, 506c, 506d, 506e, and 506f functions as a reaction zone. Each of the reaction zones yields a crude acrylate product, which exits the reaction zone via the respective exit line. Zone 506a comprises exit line 516a; zone 506b comprises exit line 516b; zone 506c comprises exit line 516c, zone 506d comprises exit line 516d, zone 506e comprises exit line 516e, and zone 506f comprises exit line 516f.

At least one of zones 506a, 506b, 506c, 506d, 506e, and 506f functions as a regeneration zone. Each regeneration zone yields a used regeneration stream, which exits the regeneration zone via the respective exit line. Zone 506a comprises exit line 517a; zone 506b comprises exit line 517b; zone 506c comprises exit line 517c, zone 506d comprises exit line 517d, zone 506e comprises exit line 517e, and zone 506f comprises exit line 517f.

When any of zones 506a, 506b, 506c, 506d, 506e, and/or 506f function as a reaction zone, the respective exit lines 516a, 516b, 516c, 516d, 516e and/or 516f convey crude acrylate product to combined acrylate product stream 516. When any of zones 506a, 506b, 506c, 506d, 506e, and/or 506f function as a regeneration zone, no crude acrylate product exits the respective regeneration zone. When any of zones 506a, 506b, 506c, 506d, 506e, and/or 506f function as a regeneration zone, the respective exit lines 517a, 517b, 517c, 517d, 517e, and/or 517f convey used regeneration agent to combined used regeneration stream 517, which may be recycled and re-used. When any of zones 506a, 506b, 506c, 506d, 506e, and/or 506f function as a reaction zone, little or no used regeneration agent exits the respective reaction zone.

As discussed above, the reactant stream in any of the reaction zones may be discontinued and replaced with a regeneration stream, thus changing the reaction zone to a regeneration zone. The catalyst activity of any of the zones may be monitored. The changing of the zones may be based on the monitoring of the catalyst activity.

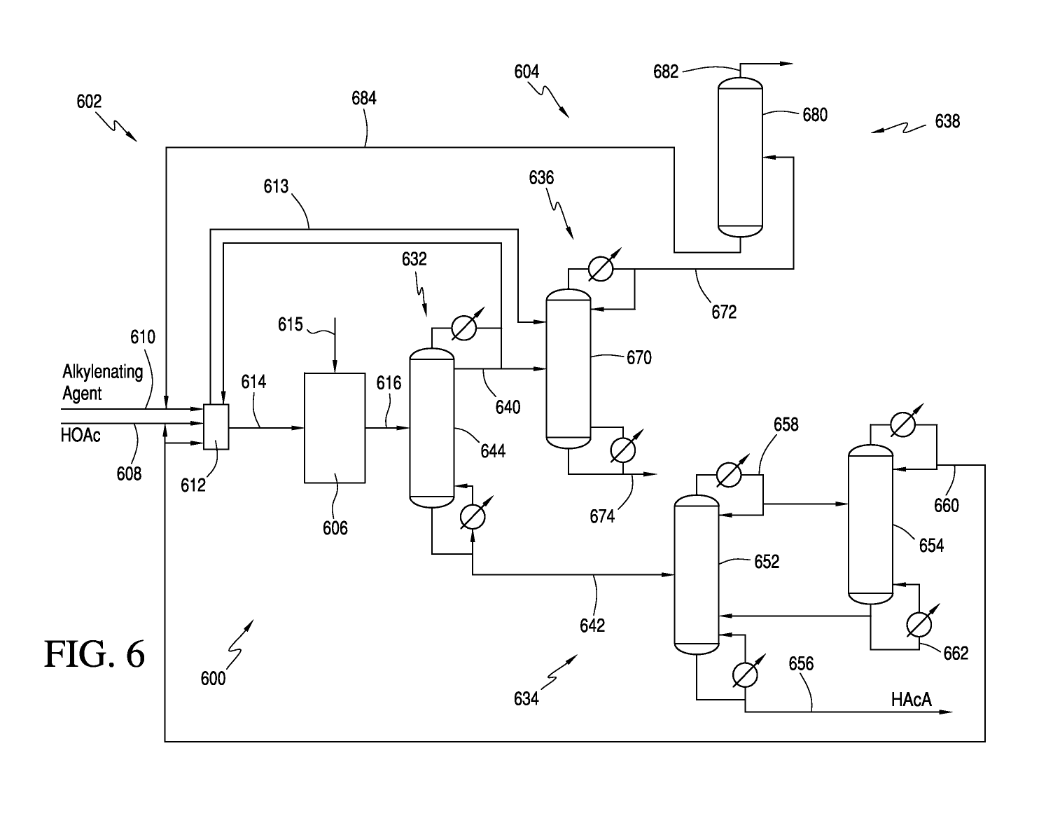

FIG. 6 shows an overview of a reaction/separation scheme in accordance with the present invention. Acrylate product system 600 comprises reaction scheme 602 and separation scheme 604. Reaction scheme 602 comprises general reaction system 606, alkanoic acid feed, e.g., acetic acid feed, 608, alkylenating agent feed, e.g., formaldehyde feed, 610, optional oxygen feed (not shown), optional methanol feed (not shown), vaporizer 612, and line 614. Reaction scheme 602 and the components thereof function in a manner similar to reaction scheme 102 of FIG. 1.

In one example, separation scheme 604 contains multiple columns, as shown in FIG. 6. Separation scheme 604 comprises alkylenating agent split unit 632, acrylate product split unit 634, drying unit 636, and methanol removal unit 638. In one embodiment, the inventive process comprises the step of separating at least a portion of the crude acrylate product to form an alkylenating agent stream and an intermediate product stream. This separating step may be referred to as the “alkylenating agent split.”

Exemplary compositional ranges for the intermediate acrylate product stream are shown in Table 2. Components other than those listed in Table 2 may also be present in the intermediate acrylate product stream. Examples include methanol, methyl acetate, methyl acrylate, dimethyl ketone, carbon dioxide, carbon monoxide, oxygen, nitrogen, and acetone.

TABLE 2
INTERMEDIATE ACRYLATE PRODUCT STREAM COMPOSITION

| | Conc. (wt. %) | Conc. (wt. %) | Conc. (wt. %) |
|---|---|---|---|
| Acrylic Acid | at least 5 | 5 to 99 | 35 to 65 |
| Acetic Acid | less than 95 | 5 to 90 | 20 to 60 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 to 1 |

In one embodiment, the alkylenating agent stream comprises significant amounts of alkylenating agent(s). For example, the alkylenating agent stream may comprise at least 1 wt. % alkylenating agent(s), e.g., at least 5 wt. %, at least 10 wt. %, at least 15 wt. %, or at least 25 wt. %. In terms of ranges, the alkylenating stream may comprise from 1 wt. % to 75 wt. % alkylenating agent(s), e.g., from 3 to 50 wt. %, from 3 wt. % to 25 wt. %, or from 10 wt. % to 20 wt. %. In terms of upper limits, the alkylenating stream may comprise less than 75 wt. % alkylenating agent(s), e.g. less than 50 wt. % or less than 40 wt. %. In preferred embodiments, the alkylenating agent is formaldehyde.

As noted above, the presence of alkylenating agent in the crude acrylate product adds unpredictability and problems to separation schemes. Without being bound by theory, it is believed that formaldehyde reacts in many side reactions with water to form by-products. The following side reactions are exemplary.

CH₂O+H₂O→>HOCH₂OH

HO(CH₂O)ᵢ₋₁H+HOCH₂OH→HO(CH₂O)ᵢH+H₂O for i>1

Without being bound by theory, it is believed that, in some embodiments, as a result of these reactions, the alkylenating agent, e.g., formaldehyde, acts as a “light” component at higher temperatures and as a “heavy” component at lower temperatures. The reaction(s) are exothermic. Accordingly, the equilibrium constant increases as temperature decreases and decreases as temperature increases. At lower temperatures, the larger equilibrium constant favors methylene glycol and oligomer production and formaldehyde becomes limited, and, as such, behaves as a heavy component. At higher temperatures, the smaller equilibrium constant favors formaldehyde production and methylene glycol becomes limited. As such, formaldehyde behaves as a light component. In view of these difficulties, as well as others, the separation of streams that comprise water and formaldehyde cannot be expected to behave as a typical two-component system. These features contribute to the unpredictability and difficulty of the separation of the unique crude acrylate product of the present invention.

The present invention, surprisingly and unexpectedly, achieves effective separation of alkylenating agent(s) from the inventive crude acrylate product to yield a purified product comprising acrylate product and very low amounts of other impurities.

In one embodiment, the alkylenating split is performed such that a lower amount of acetic acid is present in the resulting alkylenating stream. Preferably, the alkylenating agent stream comprises little or no acetic acid. As an example, the alkylenating agent stream, in some embodiments, comprises less than 50 wt. % acetic acid, e.g., less than 45 wt. %, less than 25 wt. %, less than 10 wt. %, less than 5 wt. %, less than 3 wt. %, or less than 1 wt. %. Surprisingly and unexpectedly, the present invention provides for the lower amounts of acetic acid in the alkylenating agent stream, which, beneficially reduces or eliminates the need for further treatment of the alkylenating agent stream to remove acetic acid. In some embodiments, the alkylenating agent stream may be treated to remove water therefrom, e.g., to purge water.

In some embodiments, the alkylenating agent split is performed in at least one column, e.g., at least two columns or at least three columns. Preferably, the alkylenating agent is performed in a two column system. In other embodiments, the alkylenating agent split is performed via contact with an extraction agent. In other embodiments, the alkylenating agent split is performed via precipitation methods, e.g., crystallization, and/or azeotropic distillation. Of course, other suitable separation methods may be employed either alone or in combination with the methods mentioned herein.

The intermediate product stream comprises acrylate products. In one embodiment, the intermediate product stream comprises a significant portion of acrylate products, e.g., acrylic acid. For example, the intermediate product stream may comprise at least 5 wt. % acrylate products, e.g., at least 25 wt. %, at least 40 wt. %, at least 50 wt. %, or at least 60 wt. %. In terms of ranges, the intermediate product stream may comprise from 5 wt. % to 99 wt. % acrylate products, e.g. from 10 wt. % to 90 wt. %, from 25 wt. % to 75 wt. %, or from 35 wt. % to 65 wt. %. The intermediate product stream, in one embodiment, comprises little if any alkylenating agent. For example, the intermediate product stream may comprise less than 1 wt. % alkylenating agent, e.g., less than 0.1 wt. % alkylenating agent, less than 0.05 wt. %, or less than 0.01 wt. %. In addition to the acrylate products, the intermediate product stream optionally comprises acetic acid, water, propionic acid and other components.

In some cases, the intermediate acrylate product stream comprises higher amounts of alkylenating agent. For example, in one embodiment, the intermediate acrylate product stream comprises from 1 wt. % to 50 wt. % alkylenating agent, e.g., from 1 wt. % to 10 wt. % or from 5 wt. % to 50 wt. %. In terms of limits, the intermediate acrylate product stream may comprise at least 1 wt. % alkylenating agent, e.g., at least 5 wt. % or at least 10 wt. %.

In one embodiment, the crude acrylate product is optionally treated, e.g., separated, prior to the separation of alkylenating agent therefrom. In such cases, the treatment(s) occur before the alkylenating agent split is performed. In other embodiments, at least a portion of the intermediate acrylate product stream may be further treated after the alkylenating agent split. As one example, the crude acrylate product may be treated to remove light ends therefrom. This treatment may occur either before or after the alkylenating agent split, preferably before the alkylenating agent split. In some of these cases, the further treatment of the intermediate acrylate product stream may result in derivative streams that may be considered to be additional purified acrylate product streams. In other embodiments, the further treatment of the intermediate acrylate product stream results in at least one finished acrylate product stream.

In one embodiment, the inventive process operates at a high process efficiency. For example, the process efficiency may be at least 10%, e.g., at least 20% or at least 35%. In one embodiment, the process efficiency is calculated based on the flows of reactants into the reaction zone. The process efficiency may be calculated by the following formula.

Process Efficiency=2NHAcA/[NHOAc+NHCHO+NH2O]

where:

NHAcA is the molar production rate of acrylate products; and

NHOAc, NHCHO, and NH2O are the molar feed rates of acetic acid, formaldehyde, and water.

In other embodiments, the intermediate acrylate product stream comprises higher amounts of alkylenating agent. For example, the intermediate acrylate product stream may comprise from 1 wt. % to 10 wt. % alkylenating agent, e.g., from 1 wt. % to 8 wt. % or from 2 wt. % to 5 wt. %. In one embodiment, the intermediate acrylate product stream comprises greater than 1 wt. % alkylenating agent, e.g., greater than 5 wt. % or greater than 10 wt. %.

Exemplary compositional ranges for the alkylenating agent stream are shown in Table 3. Components other than those listed in Table 3 may also be present in the purified alkylate product stream. Examples include methanol, methyl acetate, methyl acrylate, dimethyl ketone, carbon dioxide, carbon monoxide, oxygen, nitrogen, and acetone.

TABLE 3
ALKYLENATING AGENT STREAM COMPOSITION

| | Conc. (wt. %) | Conc. (wt. %) | Conc. (wt. %) |
|---|---|---|---|
| Acrylic Acid | less than 15 | 0.01 to 10 | 0.1 to 5 |
| Acetic Acid | 10 to 65 | 20 to 65 | 25 to 55 |
| Water | 15 to 75 | 25 to 65 | 30 to 60 |
| Alkylenating Agent | at least 1 | 1 to 75 | 10 to 20 |
| Propionic Acid | <10 | 0.001 to 5 | 0.01 to 1 |

In other embodiments, the alkylenating stream comprises lower amounts of acetic acid. For example, the alkylenating agent stream may comprise less than 10 wt. % acetic acid, e.g., less than 5 wt. % or less than 1 wt. %.

As mentioned above, the crude acrylate product of the present invention comprises little, if any, furfural and/or acrolein. As such the derivative stream(s) of the crude acrylate products will comprise little, if any, furfural and/or acrolein. In one embodiment, the derivative stream(s), e.g., the streams of the separation zone, comprises less than less than 500 wppm acrolein, e.g., less than 100 wppm, less than 50 wppm, or less than 10 wppm. In one embodiment, the derivative stream(s) comprises less than less than 500 wppm furfural, e.g., less than 100 wppm, less than 50 wppm, or less than 10 wppm.

Separation scheme 604 may also comprise a light ends removal unit. For example, the light ends removal unit may comprise a condenser and/or a flasher. The light ends removal unit may be configured either upstream of the alkylenating agent split unit. Depending on the configuration, the light ends removal unit removes light ends from the crude acrylate product, the alkylenating stream, and/or the intermediate acrylate product stream. In one embodiment, when the light ends are removed, the remaining liquid phase comprises the acrylic acid, acetic acid, alkylenating agent, and/or water.

Alkylenating agent split unit 632 may comprise any suitable separation device or combination of separation devices. For example, alkylenating agent split unit 632 may comprise a column, e.g., a standard distillation column, an extractive distillation column and/or an azeotropic distillation column. In other embodiments, alkylenating agent split unit 632 comprises a precipitation unit, e.g., a crystallizer and/or a chiller. Preferably, alkylenating agent split unit 632 comprises a single distillation column.

In another embodiment, the alkylenating agent split is performed by contacting the crude acrylate product with a solvent that is immiscible with water. For example, alkylenating agent split unit 632 may comprise at least one liquid-liquid extraction column. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillations, e.g., standard distillation, and crystallization. Of course, other suitable separation devices may be employed either alone or in combination with the devices mentioned herein.

In FIG. 6, alkylenating agent split unit 632 comprises first column 644. The crude acrylate product in line 616 is directed to first column 644. The crude acrylate product in line 616, in some embodiments, may be a combined crude acrylate product as indicated by elements 216, 316, 416, and 516 in the previous FIGS. First column 644 separates the crude acrylate product to form a distillate in line 640 and a residue in line 642. The distillate may be refluxed and the residue may be boiled up as shown. Stream 640 comprises at least 1 wt % alkylenating agent. As such, stream 640 may be considered an alkylenating agent stream. The first column residue exits first column 644 in line 642 and comprises a significant portion of acrylate product. As such, stream 642 is an intermediate product stream. In one embodiment, at least a portion of stream 640 is directed to drying column 636.

Exemplary compositional ranges for the distillate and residue of first column 644 are shown in Table 4. Components other than those listed in Table 4 may also be present in the residue and distillate.

TABLE 4
FIRST COLUMN

| | Conc. (wt. %) | Conc. (wt. %) | Conc. (wt. %) |
|---|---|---|---|
| Distillate | | | |
| Acrylic Acid | less than 5 | less than 3 | 0.05 to 1 |
| Acetic Acid | less than 10 | less than 5 | 0.5 to 3 |
| Water | 40 to 90 | 45 to 85 | 50 to 80 |
| Alkylenating Agent | at least 1 | 1 to 75 | 10 to 40 |
| Propionic Acid | less than 10 | less than 5 | less than 1 |
| Methanol | less than 5 | less than 1 | less than 0.5 |
| Residue | | | |
| Acrylic Acid | 10 to 80 | 15 to 65 | 20 to 50 |
| Acetic Acid | 40 to 80 | 45 to 70 | 50 to 65 |
| Water | 1 to 40 | 1 to 20 | 1 to 10 |
| Alkylenating Agent | at least 1 | 1 to 50 | 1 to 10 |
| Propionic Acid | less than 10 | less than 5 | less than 1 |

In one embodiment, the first distillate comprises smaller amounts of acetic acid, e.g., less than 25 wt %, less than 10 wt %, e.g., less than 5 wt % or less than 1 wt %. In one embodiment, the first residue comprises larger amounts of alkylenating agent.

In some embodiments, the intermediate acrylate product stream comprises higher amounts of alkylenating agent, e.g., greater than 1 wt % greater than 5 wt % or greater than 10 wt %.

For convenience, the distillate and residue of the first column may also be referred to as the “first distillate” or “first residue.” The distillates or residues of the other columns may also be referred to with similar numeric modifiers (second, third, etc.) in order to distinguish them from one another, but such modifiers should not be construed as requiring any particular separation order.

In one embodiment, polymerization inhibitors and/or anti-foam agents may be employed in the separation zone, e.g., in the units of the separation zone. The inhibitors may be used to reduce the potential for fouling caused by polymerization of acrylates. The anti-foam agents may be used to reduce potential for foaming in the various streams of the separation zone. The polymerization inhibitors and/or the anti-foam agents may be used at one or more locations in the separation zone.

In cases where any of alkylenating agent split unit 632 comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 70 kPa, e.g., from 10 kPa to 100 kPa or from 40 kPa to 80 kPa. In preferred embodiments, the pressure at which the column(s) are operated is kept at a low level e.g., less than 100 kPa, less than 80 kPa, or less than 60 kPa. In terms of lower limits, the column(s) may be operated at a pressures of at least 1 kPa, e.g., at least 20 kPa or at least 40 kPa. Without being bound by theory, it is believed that alkylenating agents, e.g., formaldehyde, may not be sufficiently volatile at lower pressures. Thus, maintenance of the column pressures at these levels surprisingly and unexpectedly provides for efficient separation operations. In addition, it has surprisingly and unexpectedly been found that by maintaining a low pressure in the columns of alkylenating agent split unit 632 may inhibit and/or eliminate polymerization of the acrylate products, e.g., acrylic acid, which may contribute to fouling of the column(s).

In one embodiment, the alkylenating agent split is achieved via one or more liquid-liquid extraction units. Preferably, the one or more liquid-liquid extraction units employ one or more extraction agents. Multiple liquid-liquid extraction units may be employed to achieve the alkylenating agent split. Any suitable liquid-liquid extraction devices used for multiple equilibrium stage separations may be used. Also, other separation devices, e.g., traditional columns, may be employed in conjunction with the liquid-liquid extraction unit(s).

In one embodiment (not shown), the crude acrylate product is fed to a liquid-liquid extraction column where the crude acrylate product is contacted with an extraction agent, e.g., an organic solvent. The liquid-liquid extraction column extracts the acids, e.g., acrylic acid and acetic acid, from the crude acrylate product. An aqueous phase comprising water, alkylenating agent, and some acetic acid exits the liquid-liquid extraction unit. Small amounts of acrylic acid may also be present in the aqueous stream. The aqueous phase may be further treated and/or recycled. An organic phase comprising acrylic acid, acetic acid, and the extraction agent also exits the liquid-liquid extraction unit. The organic phase may also comprise water and formaldehyde. The acrylic acid may be separated from the organic phase and collected as product. The acetic acid may be separated then recycled and/or used elsewhere. The solvent may be recovered and recycled to the liquid-liquid extraction unit.

The inventive process further comprises the step of separating the intermediate acrylate product stream to form a finished acrylate product stream and a first finished acetic acid stream. The finished acrylate product stream comprises acrylate product(s) and the first finished acetic acid stream comprises acetic acid. The separation of the acrylate products from the intermediate product stream to form the finished acrylate product may be referred to as the “acrylate product split.”

Returning to FIG. 6, intermediate product stream 642 exits alkylenating agent split unit 632 and is directed to acrylate product split unit 634 for further separation, e.g., to further separate the acrylate products therefrom. Acrylate product split unit 634 may comprise any suitable separation device or combination of separation devices. For example, acrylate product split unit 634 may comprise at least one column, e.g., a standard distillation column, an extractive distillation column and/or an azeotropic distillation column. In other embodiments, acrylate product split unit 634 comprises a precipitation unit, e.g., a crystallizer and/or a chiller. Preferably, acrylate product split unit 634 comprises two standard distillation columns as shown in FIG. 6. In another embodiment, acrylate product split unit 634 comprises a liquid-liquid extraction unit. Of course, other suitable separation devices may be employed either alone or in combination with the devices mentioned herein.

In FIG. 6, acrylate product split unit 634 comprises second column 652 and third column 654. Acrylate product split unit 634 receives at least a portion of intermediate acrylic product stream in line 642 and separates same into finished acrylate product stream 656 and at least one acetic acid-containing stream. As such, acrylate product split unit 634 may yield the finished acrylate product.

As shown in FIG. 6, at least a portion of intermediate acrylic product stream in line 642 is directed to second column 652. Second column 652 separates the purified acrylic product stream to form second distillate, e.g., line 658, and second residue, which is the finished acrylate product stream, e.g., line 656. The distillate may be refluxed and the residue may be boiled up as shown.

Stream 658 comprises acetic acid and some acrylic acid. The second column residue exits second column 652 in line 656 and comprises a significant portion of acrylate product. As such, stream 656 is a finished product stream. Exemplary compositional ranges for the distillate and residue of second column 652 are shown in Table 5. Components other than those listed in Table 5 may also be present in the residue and distillate.

TABLE 5
SECOND COLUMN

| | Conc. (wt. %) | Conc. (wt. %) | Conc. (wt. %) |
|---|---|---|---|
| Distillate | | | |
| Acrylic Acid | 0.1 to 40 | 1 to 30 | 5 to 30 |
| Acetic Acid | 60 to 99 | 70 to 90 | 75 to 85 |
| Water | 0.1 to 25 | 0.1 to 10 | 1 to 5 |
| Alkylenating Agent | 0.1 to 10 | 0.5 to 15 | 1 to 5 |
| Propionic Acid | less than 10 | 0.001 to 5 | 0.001 to 1 |
| Residue | | | |
| Acrylic Acid | at least 85 | 85 to 99.9 | 95 to 99.5 |
| Acetic Acid | less than 15 | 0.1 to 10 | 0.1 to 5 |
| Water | less than 1 | less than 0.1 | less than 0.01 |
| Alkylenating Agent | less than 1 | less than 0.1 | less than 0.01 |
| Propionic Acid | less than 1 | less than 0.1 | less than 0.01 |

Returning to FIG. 6, at least a portion of stream 658 is directed to third column 654. Third column 654 separates the at least a portion of stream 658 into a distillate in line 660 and a residue in line 662. The distillate may be refluxed and the residue may be boiled up as shown. The distillate comprises a major portion of acetic acid. In one embodiment, at least a portion of line 660 is returned, either directly or indirectly, to reaction system 606. The third column residue exits third column 654 in line 662 and comprises acetic acid and some acrylic acid. At least a portion of line 662 may be returned to second column 652 for further separation. In one embodiment, at least a portion of line 662 is returned, either directly or indirectly, to reaction system 606. In another embodiment, at least a portion of the acetic acid-containing stream in either or both of lines 660 and 662 may be directed to an ethanol production system that utilizes the hydrogenation of acetic acid to form the ethanol. In another embodiment, at least a portion of the acetic acid-containing stream in either or both of lines 660 and 662 may be directed to a vinyl acetate system that utilizes the reaction of ethylene, acetic acid, and oxygen form the vinyl acetate. Exemplary compositional ranges for the distillate and residue of third column 654 are shown in Table 6. Components other than those listed in Table 6 may also be present in the residue and distillate.

TABLE 6
THIRD COLUMN

| | Conc. (wt. %) | Conc. (wt. %) | Conc. (wt. %) |
|---|---|---|---|
| Distillate | | | |
| Acrylic Acid | 0.01 to 10 | 0.05 to 5 | 0.1 to 1 |
| Acetic Acid | 50 to 99.9 | 70 to 99.5 | 80 to 99 |
| Water | 0.1 to 25 | 0.1 to 15 | 1 to 10 |
| Alkylenating Agent | 0.1 to 25 | 0.1 to 15 | 1 to 10 |
| Propionic Acid | less than 1 | less than 0.1 | less than 0.01 |
| Residue | | | |
| Acrylic Acid | 5 to 50 | 15 to 40 | 20 to 35 |
| Acetic Acid | 50 to 95 | 60 to 80 | 65 to 75 |
| Water | 0.01 to 10 | 0.01 to 5 | 0.1 to 1 |
| Alkylenating Agent | less than 1 | 0.001 to 1 | 0.01 to 1 |
| Propionic Acid | less than 1 | less than 0.1 | less than 0.01 |

In cases where the acrylate product split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 70 kPa, e.g., from 10 kPa to 100 kPa or from 40 kPa to 80 kPa. In preferred embodiments, the pressure at which the column(s) are operated is kept at a low level e.g., less than 50 kPa, less than 27 kPa, or less than 20 kPa. In terms of lower limits, the column(s) may be operated at a pressures of at least 1 kPa, e.g., at least 3 kPa or at least 5 kPa. Without being bound by theory, it has surprisingly and unexpectedly been found that be maintaining a low pressure in the columns of acrylate product split unit 634 may inhibit and/or eliminate polymerization of the acrylate products, e.g., acrylic acid, which may contribute to fouling of the column(s).

It has also been found that, surprisingly and unexpectedly, maintaining the temperature of acrylic acid-containing streams fed to acrylate product split unit 634 at temperatures below 140° C., e.g., below 130° C. or below 115° C., may inhibit and/or eliminate polymerization of acrylate products. In one embodiment, to maintain the liquid temperature at these temperatures, the pressure of the column(s) is maintained at or below the pressures mentioned above. In these cases, due to the lower pressures, the number of theoretical column trays is kept at a low level, e.g., less than 10, less than 8, less than 7, or less than 5. As such, it has surprisingly and unexpectedly been found that multiple columns having fewer trays inhibit and/or eliminate acrylate product polymerization. In contrast, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns each may operate at the lower pressures discussed above.

Returning to FIG. 6, alkylenating agent stream 640 exits alkylenating agent split unit 632 and is directed to drying unit 636 for further separation, e.g., to further separate the water therefrom. The separation of the formaldehyde from the water may be referred to as dehydration. Drying unit 636 may comprise any suitable separation device or combination of separation devices. For example, drying unit 636 may comprise at least one column, e.g., a standard distillation column, an extractive distillation column and/or an azeotropic distillation column. In other embodiments, drying unit 636 comprises a dryer and/or a molecular sieve unit. In a preferred embodiment, drying unit 636 comprises a liquid-liquid extraction unit. In one embodiment, drying unit 636 comprises a standard distillation column as shown in FIG. 6. Of course, other suitable separation devices may be employed either alone or in combination with the devices mentioned herein.

In FIG. 6, drying unit 636 comprises fourth column 670. Drying unit 636 receives at least a portion of alkylenating agent stream in line 640 and separates same into a fourth distillate comprising water, formaldehyde, and methanol in line 672 and a fourth residue comprising mostly water in line 674. The distillate may be refluxed and the residue may be boiled up as shown. In one embodiment, at least a portion of line 672 is returned, either directly or indirectly, to reaction system 606.

Exemplary compositional ranges for the distillate and residue of fourth column 670 are shown in Table 7. Components other than those listed in Table 7 may also be present in the residue and distillate.

TABLE 7
FOURTH COLUMN

| | Conc. (wt. %) | Conc. (wt. %) | Conc. (wt. %) |
|---|---|---|---|
| Distillate | | | |
| Acrylic Acid | less than 1 | less than 0.1 | less than 0.01 |
| Acetic Acid | less than 2 | 0.01 to 1 | 0.01 to 1 |
| Water | 20 to 90 | 30 to 80 | 40 to 70 |
| Alkylenating Agent | 10 to 70 | 20 to 60 | 30 to 50 |
| Methanol | 0.01 to 15 | 0.1 to 10 | 1 to 5 |
| Residue | | | |
| Acrylic Acid | less than 1 | 0.001 to 1 | 0.01 to 1 |
| Acetic Acid | less than 15 | 0.1 to 10 | 0.1 to 5 |
| Water | at least 85 | 85 to 99.9 | 95 to 99.5 |
| Alkylenating Agent | less than 1 | 0.001 to 1 | 0.1 to 1 |
| Propionic Acid | less than 1 | less than 0.1 | less than 0.01 |

In cases where the drying unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 70 kPa.

Returning to FIG. 6, alkylenating agent stream 672 exits drying unit 636 and is directed to methanol removal unit 638 for further separation, e.g., to further separate the methanol therefrom. Methanol removal unit 638 may comprise any suitable separation device or combination of separation devices. For example, methanol removal unit 638 may comprise at least one column, e.g., a standard distillation column, an extractive distillation column and/or an azeotropic distillation column. In one embodiment, methanol removal unit 638 comprises a liquid-liquid extraction unit. In a preferred embodiment, methanol removal unit 638 comprises a standard distillation column as shown in FIG. 6. Of course, other suitable separation devices may be employed either alone or in combination with the devices mentioned herein.

In FIG. 6, methanol removal unit 638 comprises fifth column 680. Methanol removal unit 638 receives at least a portion of line 672 and separates same into a fifth distillate comprising methanol and water in line 682 and a fifth residue comprising water and formaldehyde in line 684. The distillate may be refluxed and the residue may be boiled up (not shown). In one embodiment, at least a portion of line 684 is returned, either directly or indirectly, to reaction system 606. Fifth distillate 682, for example, may be used to form additional formaldehyde.

Exemplary compositional ranges for the distillate and residue of fifth column 680 are shown in Table 8. Components other than those listed in Table 8 may also be present in the residue and distillate.

TABLE 8
FIFTH COLUMN

| | Conc. (wt. %) | Conc. (wt. %) | Conc. (wt. %) |
|---|---|---|---|
| Distillate | | | |
| Acrylic Acid | less than 1 | less than 0.1 | less than 0.01 |
| Acetic Acid | less than 1 | less than 0.1 | less than 0.01 |
| Water | 20 to 60 | 30 to 50 | 35 to 45 |
| Alkylenating Agent | 0.1 to 25 | 0.5 to 20 | 1 to 15 |
| Methanol | 20 to 70 | 30 to 60 | 40 to 50 |
| Residue | | | |
| Acrylic Acid | less than 1 | less than 0.1 | less than 0.01 |
| Acetic Acid | less than 15 | 0.1 to 10 | 0.1 to 5 |
| Water | 40 to 80 | 50 to 70 | 55 to 65 |
| Alkylenating Agent | 20 to 60 | 30 to 50 | 35 to 45 |
| Methanol | less than 15 | 0.1 to 10 | 0.1 to 5 |

In cases where the methanol removal unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 70 kPa.

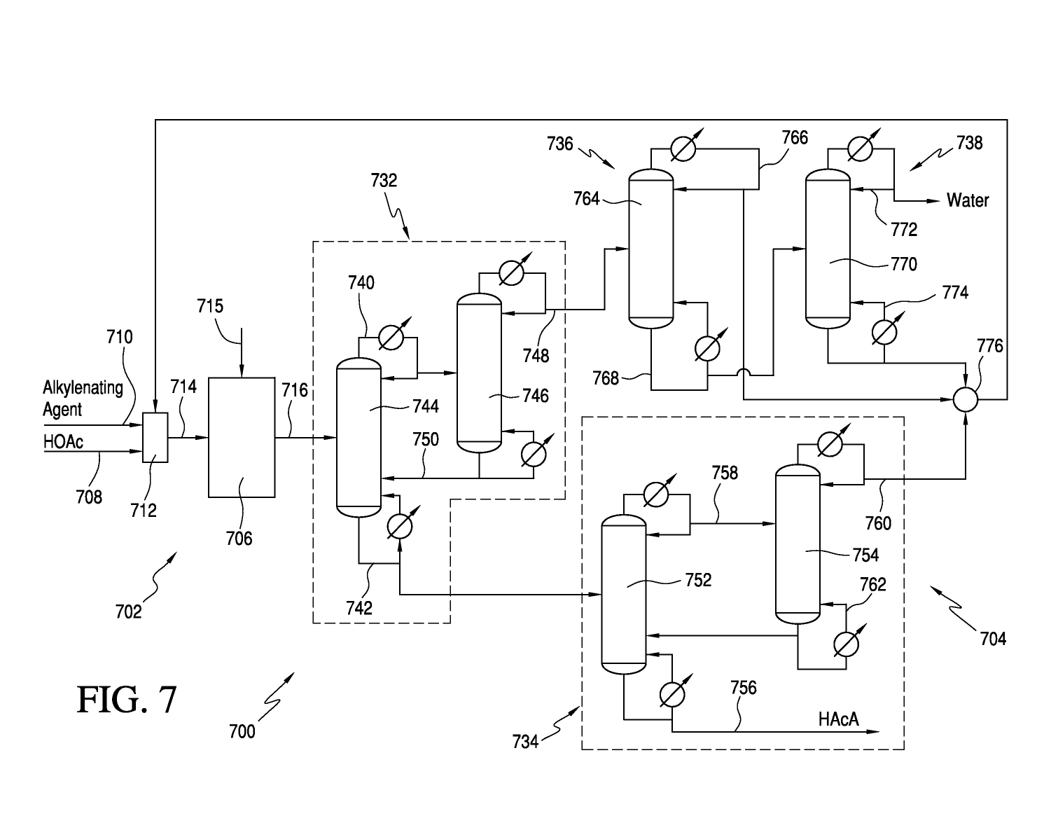

FIG. 7 shows an overview of a reaction/separation scheme in accordance with the present invention. Acrylate product system 700 comprises reaction scheme 702 and separation scheme 704. Reaction scheme 702 comprises general reaction system 706, alkanoic acid feed, e.g., acetic acid feed, 708, alkylenating agent feed, e.g., formaldehyde feed, 710, optional oxygen feed (not shown), optional methanol feed (not shown), vaporizer 712, and line 714. Reaction scheme 702 and the components thereof function in a manner similar to reaction scheme 102 of FIG. 1.

Reaction scheme 702 yields a crude acrylate product, which exits reaction scheme 702 via line 716 and is directed to separation scheme 704. The crude acrylate product in line 716, in some embodiments, may be a combined crude acrylate product as indicated by elements 216, 316, 416, and 516 in the previous FIGS. The components of the crude acrylate product are discussed above. Separation scheme 704 comprises alkylenating agent split unit 732, acrylate product split unit 734, acetic acid split unit 736, and drying unit 738. Separation scheme 704 may also comprise an optional light ends removal unit (not shown). For example, the light ends removal unit may comprise a condenser and/or a flasher. The light ends removal unit may be configured either upstream or downstream of the alkylenating agent split unit. Depending on the configuration, the light ends removal unit removes light ends from the crude acrylate product, the alkylenating stream, and/or the intermediate acrylate product stream. In one embodiment, when the light ends are removed, the remaining liquid phase comprises the acrylic acid, acetic acid, alkylenating agent, and/or water.

Alkylenating agent split unit 732 may comprise any suitable separation device or combination of separation devices. For example, alkylenating agent split unit 732 may comprise a column, e.g., a standard distillation column, an extractive distillation column and/or an azeotropic distillation column. In other embodiments, alkylenating agent split unit 732 comprises a precipitation unit, e.g., a crystallizer and/or a chiller. Preferably, alkylenating agent split unit 732 comprises two standard distillation columns. In another embodiment, the alkylenating agent split is performed by contacting the crude acrylate product with a solvent that is immiscible with water. For example alkylenating agent split unit 732 may comprise at least one liquid-liquid extraction columns. In another embodiment, the alkylenating agent split is performed via azeotropic distillation, which employs an azeotropic agent. In these cases, the azeotropic agent may be selected from the group consisting of methyl isobutylketene, o-xylene, toluene, benzene, n-hexane, cyclohexane, p-xylene, and mixtures thereof. This listing is not exclusive and is not meant to limit the scope of the invention. In another embodiment, the alkylenating agent split is performed via a combination of distillation, e.g., standard distillation, and crystallization. Of course, other suitable separation devices may be employed either alone or in combination with the devices mentioned herein.

In FIG. 7, alkylenating agent split unit 732 comprises sixth column 744 and seventh column 746. Alkylenating agent split unit 732 receives crude acrylic product stream in line 716 and separates same into at least one alkylenating agent stream, e.g., stream 748, and at least one purified product stream, e.g., stream 742. Alkylenating agent split unit 732 performs an alkylenating agent split, as discussed above.

In operation, as shown in FIG. 7, the crude acrylate product in line 716 is directed to sixth column 744. Sixth column 744 separates the crude acrylate product a distillate in line 740 and a residue in line 742. The distillate may be refluxed and the residue may be boiled up as shown. Stream 740 comprises at least 1 wt % alkylenating agent. As such, stream 740 may be considered an alkylenating agent stream. The sixth column residue exits sixth column 744 in line 742 and comprises a significant portion of acrylate product. As such, stream 742 is an intermediate product stream. Exemplary compositional ranges for the distillate and residue of sixth column 744 are shown in Table 9. Components other than those listed in Table 9 may also be present in the residue and distillate.

TABLE 9
SIXTH COLUMN

| | Conc. (wt. %) | Conc. (wt. %) | Conc. (wt. %) |
|---|---|---|---|
| Distillate | | | |
| Acrylic Acid | 0.1 to 20 | 1 to 10 | 1 to 5 |
| Acetic Acid | 25 to 65 | 35 to 55 | 40 to 50 |
| Water | 15 to 55 | 25 to 45 | 30 to 40 |
| Alkylenating Agent | at least 1 | 1 to 75 | 10 to 20 |
| Propionic Acid | <10 | 0.001 to 5 | 0.001 to 1 |
| Residue | | | |
| Acrylic Acid | at least 5 | 5 to 99 | 35 to 65 |
| Acetic Acid | less than 95 | 5 to 90 | 20 to 60 |
| Water | less than 25 | 0.1 to 10 | 0.5 to 7 |
| Alkylenating Agent | <1 | <0.5 | <0.1 |
| Propionic Acid | <10 | 0.01 to 5 | 0.01 o 1 |

In one embodiments, the sixth distillate comprises smaller amounts of acetic acid, e.g., less than 25 wt %, less than 10 wt %, e.g., less than 5 wt % or less than 1 wt %. In one embodiment, the first residue comprises larger amounts of alkylenating agent, e.g.,

In other embodiments, the intermediate acrylate product stream comprises higher amounts of alkylenating agent, e.g., greater than 1 wt % greater than 5 wt % or greater than 10 wt %.

Returning to FIG. 7, at least a portion of stream 740 is directed to seventh column 746. Seventh column 746 separates the at least a portion of stream 740 into a distillate in line 748 and a residue in line 750. The distillate may be refluxed and the residue may be boiled up as shown. The distillate comprises at least 1 wt % alkylenating agent. Stream 748, like stream 740, may be considered an alkylenating agent stream. The seventh column residue exits seventh column 746 in line 750 and comprises a significant portion of acetic acid. At least a portion of line 750 may be returned to sixth column 744 for further separation. In one embodiment, at least a portion of line 750 is returned, either directly or indirectly, to reaction system 706. Exemplary compositional ranges for the distillate and residue of seventh column 746 are shown in Table 10. Components other than those listed in Table 10 may also be present in the residue and distillate.

TABLE 10
SEVENTH COLUMN

| | Conc. (wt. %) | Conc. (wt. %) | Conc. (wt. %) |
|---|---|---|---|
| Distillate | | | |
| Acrylic Acid | 0.01 to 10 | 0.05 to 5 | 0.1 to 0.5 |
| Acetic Acid | 10 to 50 | 20 to 40 | 25 to 35 |
| Water | 35 to 75 | 45 to 65 | 50 to 60 |
| Alkylenating Agent | at least 1 | 1 to 75 | 10 to 20 |
| Propionic Acid | 0.01 to 10 | 0.01 to 5 | 0.01 to 0.05 |
| Residue | | | |
| Acrylic Acid | 0.1 to 25 | 0.05 to 15 | 1 to 10 |
| Acetic Acid | 40 to 80 | 50 to 70 | 55 to 65 |
| Water | 1 to 40 | 5 to 35 | 10 to 30 |
| Alkylenating Agent | at least 1 | 1 to 75 | 10 to 20 |
| Propionic Acid | <10 | 0.001 to 5 | 0.01 1 |

As shown in FIG. 7, acrylic product stream in line 742 exits alkylenating agent split unit 732 and is directed to acrylate product split unit 734 for further separation, e.g., to further separate the acrylate products therefrom. Acrylate product split unit 734 may comprise any suitable separation device or combination of separation devices. For example, acrylate product split unit 734 may comprise at least one column, e.g., a standard distillation column, an extractive distillation column and/or an azeotropic distillation column. In other embodiments, acrylate product split unit 734 comprises a precipitation unit, e.g., a crystallizer and/or a chiller. Preferably, acrylate product split unit 734 comprises two standard distillation columns as shown in FIG. 7. In another embodiment, acrylate product split unit 734 comprises a liquid-liquid extraction unit. Of course, other suitable separation devices may be employed either alone or in combination with the devices mentioned herein.

In FIG. 7, acrylate product split unit 734 comprises eighth column 752 and ninth column 754. Acrylate product split unit 734 receives at least a portion of acrylic product stream in line 742 and separates same into finished acrylate product stream 756 and at least one acetic acid-containing stream. As such, acrylate product split unit 734 may yield the finished acrylate product.

As shown in FIG. 7, at least a portion of acrylic product stream in line 742 is directed to eighth column 752. Eighth column 752 separates the acrylic product stream to form eighth distillate, e.g., line 758, and eighth residue, which is the finished acrylate product stream, e.g., line 756. The distillate may be refluxed and the residue may be boiled up as shown.

Stream 758 comprises acetic acid and some acrylic acid. The eighth column residue exits eighth column 752 in line 756 and comprises a significant portion of acrylate product. As such, stream 756 is a finished product stream. Exemplary compositional ranges for the distillate and residue of eighth column 752 are shown in Table 11. Components other than those listed in Table 11 may also be present in the residue and distillate.

TABLE 11
EIGHTH COLUMN

| | Conc. (wt. %) | Conc. (wt. %) | Conc. (wt. %) |
|---|---|---|---|
| Distillate | | | |
| Acrylic Acid | 0.1 to 40 | 1 to 30 | 5 to 30 |
| Acetic Acid | 60 to 99 | 70 to 90 | 75 to 85 |
| Water | 0.1 to 25 | 0.1 to 10 | 1 to 5 |
| Alkylenating Agent | less than 1 | 0.001 to 1 | 0.1 to 1 |
| Propionic Acid | <10 | 0.001 to 5 | 0.001 to 1 |
| Residue | | | |
| Acrylic Acid | at least 85 | 85 to 99.9 | 95 to 99.5 |
| Acetic Acid | less than 15 | 0.1 to 10 | 0.1 to 5 |
| Water | less than 1 | less than 0.1 | less than 0.01 |
| Alkylenating Agent | less than 1 | 0.001 to 1 | 0.1 to 1 |
| Propionic Acid | 0.1 to 10 | 0.1 to 5 | 0.5 to 3 |

Returning to FIG. 7, at least a portion of stream 758 is directed to ninth column 754. Ninth column 754 separates the at least a portion of stream 758 into a distillate in line 760 and a residue in line 762. The distillate may be refluxed and the residue may be boiled up as shown. The distillate comprises a major portion of acetic acid. In one embodiment, at least a portion of line 760 is returned, either directly or indirectly, to reaction system 706. The ninth column residue exits ninth column 754 in line 762 and comprises acetic acid and some acrylic acid. At least a portion of line 762 may be returned to eighth column 752 for further separation. In one embodiment, at least a portion of line 762 is returned, either directly or indirectly, to reaction system 706. In another embodiment, at least a portion of the acetic acid-containing stream in either or both of lines 760 and 762 may be directed to an ethanol production system that utilizes the hydrogenation of acetic acid form the ethanol. In another embodiment, at least a portion of the acetic acid-containing stream in either or both of lines 760 and 762 may be directed to a vinyl acetate system that utilizes the reaction of ethylene, acetic acid, and oxygen form the vinyl acetate. Exemplary compositional ranges for the distillate and residue of ninth column 754 are shown in Table 12. Components other than those listed in Table 12 may also be present in the residue and distillate.

TABLE 12
NINTH COLUMN

| | Conc. (wt. %) | Conc. (wt. %) | Conc. (wt. %) |
|---|---|---|---|
| Distillate | | | |
| Acrylic Acid | 0.01 to 10 | 0.05 to 5 | 0.1 to 1 |
| Acetic Acid | 50 to 99.9 | 70 to 99.5 | 80 to 99 |
| Water | 0.1 to 25 | 0.1 to 15 | 1 to 10 |
| Alkylenating | less than 10 | 0.001 to 5 | 0.01 to 5 |
| Agent | | | |
| Propionic Acid | 0.0001 to 10 | 0.001 to 5 | 0.001 to 0.05 |
| Residue | | | |
| Acrylic Acid | 5 to 50 | 15 to 40 | 20 to 35 |
| Acetic Acid | 50 to 95 | 60 to 80 | 65 to 75 |
| Water | 0.01 to 10 | 0.01 to 5 | 0.1 to 1 |
| Alkylenating | less than 1 | 0.001 to 1 | 0.1 to 1 |
| Agent | | | |
| Propionic Acid | <10 | 0.001 to 5 | 0.001 to 1 |

In cases where the acrylate product split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 70 kPa, e.g., from 10 kPa to 100 kPa or from 40 kPa to 80 kPa. In preferred embodiments, the pressure at which the column(s) are operated is kept at a low level e.g., less than 50 kPa, less than 27 kPa, or less than 20 kPa. In terms of lower limits, the column(s) may be operated at a pressures of at least 1 kPa, e.g., at least 3 kPa or at least 5 kPa. Without being bound by theory, it has surprisingly and unexpectedly been found that be maintaining a low pressure in the columns of acrylate product split unit 734 may inhibit and/or eliminate polymerization of the acrylate products, e.g., acrylic acid, which may contribute to fouling of the column(s).

It has also been found that, surprisingly and unexpectedly, maintaining the temperature of acrylic acid-containing streams fed to acrylate product split unit 734 at temperatures below 140° C., e.g., below 130° C. or below 115° C., may inhibit and/or eliminate polymerization of acrylate products. In one embodiment, to maintain the liquid temperature at these temperatures, the pressure of the column(s) is maintained at or below the pressures mentioned above. In these cases, due to the lower pressures, the number of theoretical column trays is kept at a low level, e.g., less than 10, less than 8, less than 7, or less than 5. As such, it has surprisingly and unexpectedly been found that multiple columns having fewer trays inhibit and/or eliminate acrylate product polymerization. In contrast, a column having a higher amount of trays, e.g., more than 10 trays or more than 15 trays, would suffer from fouling due to the polymerization of the acrylate products. Thus, in a preferred embodiment, the acrylic acid split is performed in at least two, e.g., at least three, columns, each of which have less than 10 trays, e.g. less than 7 trays. These columns each may operate at the lower pressures discussed above.

Returning to FIG. 7, alkylenating agent stream 748 exits alkylenating agent split unit 732 and is directed to acetic acid split unit 736 for further separation, e.g., to further separate the alkylenating agent and the acetic acid therefrom. Acetic acid split unit 736 may comprise any suitable separation device or combination of separation devices. For example, acetic acid split unit 736 may comprise at least one column, e.g., a standard distillation column, an extractive distillation column and/or an azeotropic distillation column. In other embodiments, acetic acid split unit 736 comprises a precipitation unit, e.g., a crystallizer and/or a chiller. Preferably, acetic acid split unit 736 comprises a standard distillation column as shown in FIG. 7. In another embodiment, acetic acid split unit 736 comprises a liquid-liquid extraction unit. Of course, other suitable separation devices may be employed either alone or in combination with the devices mentioned herein.

In FIG. 7, acetic acid split unit 736 comprises tenth column 764. Acetic acid split unit 736 receives at least a portion of alkylenating agent stream in line 748 and separates same into a tenth distillate comprising alkylenating agent in line 766, e.g., a purified alkylenating stream, and a tenth residue comprising acetic acid in line 768, e.g., a purified acetic acid stream. The distillate may be refluxed and the residue may be boiled up as shown. In one embodiment, at least a portion of line 766 and/or line 768 are returned, either directly or indirectly, to reactor 306. At least a portion of stream in line 768 may be further separated. In another embodiment, at least a portion of the acetic acid-containing stream in line 768 may be directed to an ethanol production system that utilizes the hydrogenation of acetic acid form the ethanol. In another embodiment, at least a portion of the acetic acid-containing stream in line 768 may be directed to a vinyl acetate system that utilizes the reaction of ethylene, acetic acid, and oxygen form the vinyl acetate.

The stream in line 766 comprises alkylenating agent and water. The stream in line 768 comprises acetic acid and water. Exemplary compositional ranges for the distillate and residue of tenth column 764 are shown in Table 13. Components other than those listed in Table 13 may also be present in the residue and distillate.

TABLE 13
TENTH COLUMN

| | Conc. (wt. %) | Conc. (wt. %) | Conc. (wt. %) |
|---|---|---|---|
| Distillate | | | |
| Acrylic Acid | less than 1 | 0.001 to 5 | 0.001 to 1 |
| Acetic Acid | less than 1 | 0.001 to 5 | 0.001 to 1 |
| Water | 40 to 80 | 50 to 70 | 55 to 65 |
| Alkylenating Agent | 20 to 60 | 30 to 50 | 35 to 45 |
| Propionic Acid | less than 10 | 0.001 to 5 | 0.001 to 1 |
| Residue | | | |
| Acrylic Acid | less than 1 | 0.01 to 5 | 0.1 to 1 |
| Acetic Acid | 25 to 65 | 35 to 55 | 40 to 50 |
| Water | 35 to 75 | 45 to 65 | 50 to 60 |
| Alkylenating Agent | less than 1 | 0.01 to 5 | 0.1 to 1 |
| Propionic Acid | less than 10 | 0.001 to 5 | 0.01 to 1 |

In cases where the acetic acid split unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 70 kPa.

The inventive process further comprises the step of separating the purified acetic acid stream to form a second finished acetic acid stream and a water stream. The second finished acetic acid stream comprises a major portion of acetic acid, and the water stream comprises mostly water. The separation of the acetic from the water may be referred to as dehydration.

Returning to FIG. 7, tenth residue 768 exits acetic acid split unit 736 and is directed to drying unit 738 for further separation, e.g., to remove water from the acetic acid. Drying unit 738 may comprise any suitable separation device or combination of separation devices. For example, drying unit 738 may comprise at least one column, e.g., a standard distillation column, an extractive distillation column and/or an azeotropic distillation column. In other embodiments, drying unit 738 comprises a dryer and/or a molecular sieve unit. In a preferred embodiment, drying unit 738 comprises a liquid-liquid extraction unit. In one embodiment, drying unit 738 comprises a standard distillation column as shown in FIG. 7. Of course, other suitable separation devices may be employed either alone or in combination with the devices mentioned herein.

In FIG. 7, drying unit 738 comprises eleventh column 770. Drying unit 738 receives at least a portion of second finished acetic acid stream in line 768 and separates same into eleventh distillate comprising a major portion of water in line 772 and eleventh residue comprising acetic acid and small amounts of water in line 774. The distillate may be refluxed and the residue may be boiled up as shown. In one embodiment, at least a portion of line 774 is returned, either directly or indirectly, to reaction system 706. In another embodiment, at least a portion of the acetic acid-containing stream in line 774 may be directed to an ethanol production system that utilizes the hydrogenation of acetic acid form the ethanol. In another embodiment, at least a portion of the acetic acid-containing stream in line 774 may be directed to a vinyl acetate system that utilizes the reaction of ethylene, acetic acid, and oxygen form the vinyl acetate.

Exemplary compositional ranges for the distillate and residue of eleventh column 770 are shown in Table 14. Components other than those listed in Table 14 may also be present in the residue and distillate.

TABLE 14
ELEVENTH COLUMN

| | Conc. (wt. %) | Conc. (wt. %) | Conc. (wt. %) |
|---|---|---|---|
| Distillate | | | |
| Acrylic Acid | less than 1 | 0.001 to 5 | 0.001 to 1 |
| Acetic Acid | less than 1 | 0.01 to 5 | 0.01 to 1 |
| Water | 90 to 99.9 | 95 to 99.9 | 95 to 99.5 |
| Alkylenating Agent | less than 1 | 0.01 to 5 | 0.01 to 1 |
| Propionic Acid | less than 10 | 0.001 to 5 | 0.001 to 1 |
| Residue | | | |
| Acrylic Acid | less than 1 | 0.01 to 5 | 0.01 to 1 |
| Acetic Acid | 75 to 99.9 | 85 to 99.5 | 90 to 99.5 |
| Water | 25 to 65 | 35 to 55 | 40 to 50 |
| Alkylenating Agent | less than 1 | less than 0.001 | less than 0.0001 |
| Propionic Acid | less than 10 | 0.001 to 5 | 0.001 to 1 |

In cases where the drying unit comprises at least one column, the column(s) may be operated at suitable temperatures and pressures. In one embodiment, the temperature of the residue exiting the column(s) ranges from 90° C. to 130° C., e.g., from 95° C. to 120° C. or from 100° C. to 115° C. The temperature of the distillate exiting the column(s) preferably ranges from 60° C. to 90° C., e.g., from 65° C. to 85° C. or from 70° C. to 80° C. The pressure at which the column(s) are operated may range from 1 kPa to 500 kPa, e.g., from 25 kPa to 400 kPa or from 100 kPa to 70 kPa. FIG. 7 also shows tank 776, which, collects at least one of the process streams prior to recycling same to reaction system 706. Tank 776 is an optional feature. The various recycle streams that may, alternatively, be recycled directly to reaction system 706 without being collected in tank 776.

### EXAMPLES

Simulations were conducted using reaction systems configured in accordance with the present invention, e.g., wherein the ratio of the size of the reaction zones to the size of the regeneration zones is in accordance with the present invention, as shown in Table 15. The capital costs for different sized reactors were calculated using the 0.67-scale-law based on a capital cost of $20MM for a 50 m³ reactor. Catalyst cost estimates were calculated based on 30% of the reactor capital cost amortized over a 10 year period. Maintenance cost estimates were calculated based on 50% of the capital cost amortized over a 10 year period.

TABLE 15
REACTION SYSTEM CONFIGURATIONS

| | | | | |
|---|---|---|---|---|
| Number of Reaction Zones | 2 | 3 | 4 | 5 |
| Cost per Reaction Zone, $MM | 13 | 10 | 8 | 7 |
| Size per Reaction Zone, m³ | 25 | 17 | 13 | 10 |
| Total Cost of Reaction Zone(s), $MM | 25 | 29 | 32 | 34 |
| Total Size of Reaction Zone(s), m³ | 50 | 51 | 52 | 50 |
| Number of Regeneration Zones | 1 | 1 | 1 | 1 |
| Cost per Regeneration Zone, $MM | 13 | 10 | 8 | 7 |
| Size per Regeneration Zone, m³ | 25 | 17 | 13 | 10 |
| Total Cost of Regeneration Zone(s), $MM | 13 | 10 | 8 | 7 |
| Total Size of Regeneration Zone(s), m³ | 25 | 17 | 13 | 10 |
| Reaction:Regeneration | 2:1 | 3:1 | 4:1 | 5:1 |
| Size Ratio | | | | |
| Total Equipment Requirement, $MM | 38 | 39 | 40 | 41 |
| Catalyst Requirement, $MM | 9 | 8 | 8 | 7 |
| Maintenance Requirement, $MM | 19 | 19 | 20 | 20 |
| Total Burden (Equipment, Catalyst, and | 66 | 66 | 68 | 68 |
| Maintenance), $MM | | | | |
| STY | 500 | 500 | 500 | 500 |

### Comparative Examples

Simulations were conducted using reaction systems configured such that the ratio of the size of the reaction zones to the size of the regeneration zones was outside of the ranges of the present invention, as shown in Table 16.

TABLE 16
COMPARATIVE REACTION SYSTEM CONFIGURATIONS

| | | | | | | |
|---|---|---|---|---|---|---|
| Number of Reaction Zones | 1 | 6 | 7 | 8 | 9 | 10 |
| Cost per Reaction Zone, $MM | 20 | 6 | 5 | 5 | 5 | 4 |
| Size per Reaction Zone, m³ | 50 | 8 | 7 | 6 | 6 | 5 |
| Total Cost of Reaction | 20 | 36 | 35 | 40 | 45 | 40 |
| Zone(s), $MM | | | | | | |
| Total Size of Reaction | 50 | 48 | 49 | 48 | 54 | 50 |
| Zone(s), m³ | | | | | | |
| Number of Regeneration | 1 | 1 | 1 | 1 | 1 | 1 |
| Zones | | | | | | |
| Cost per Regeneration Zone, | 20 | 6 | 5 | 5 | 5 | 4 |
| $MM | | | | | | |
| Size per Regeneration Zone, | 50 | 8 | 7 | 6 | 6 | 5 |
| m³ | | | | | | |
| Total Cost of Regeneration | 20 | 6 | 5 | 5 | 5 | 4 |
| Zone(s), $MM | | | | | | |
| Total Size of Regeneration | 50 | 8 | 7 | 6 | 6 | 5 |
| Zone(s), m³ | | | | | | |
| Reaction:Regeneration | 1:1 | 6:1 | 7:1 | 8:1 | 9:1 | 10:1 |
| Size Ratio | | | | | | |
| Total Equipment | 40 | 42 | 40 | 45 | 50 | 44 |
| Requirement, $MM | | | | | | |
| Catalyst Requirement, $MM | 12 | 7 | 7 | 7 | 7 | 7 |
| Maintenance Requirement, | 20 | 21 | 22 | 22 | 23 | 24 |
| $MM | | | | | | |
| Total Burden (Equipment, | 72 | 70 | 69 | 72 | 80 | 75 |
| Catalyst, and Maintenance), | | | | | | |
| $MM | | | | | | |
| STY | 500 | 500 | 500 | 500 | 500 | 500 |

As shown in Tables 15 and 16, configurations wherein the ratio of the combined size of the reaction zones to the combined size of the regeneration zone ranged from 1:1 to 6:1, surprisingly and unexpectedly demonstrated an advantageous combination of capital investment, catalyst requirement (quantity and, as a result, cost), and maintenance requirement, while maintaining similar levels of acrylate product yield.

While the invention has been described in detail, modifications within the spirit and scope of the invention will be readily apparent to those of skill in the art. In view of the foregoing discussion, relevant knowledge in the art and references discussed above in connection with the Background and Detailed Description, the disclosures of which are all incorporated herein by reference. In addition, it should be understood that aspects of the invention and portions of various embodiments and various features recited below and/or in the appended claims may be combined or interchanged either in whole or in part. In the foregoing descriptions of the various embodiments, those embodiments which refer to another embodiment may be appropriately combined with other embodiments as will be appreciated by one of skill in the art. Furthermore, those of ordinary skill in the art will appreciate that the foregoing description is by way of example only, and is not intended to limit the invention.

We claim:

1. A process for producing an acrylate product, the process comprising the steps of:
(a) providing a reaction system comprising:
at least one reaction zone, each reaction zone having a size and comprising a respective catalyst; and
at least one regeneration zone, each regeneration zone having a size and comprising a respective catalyst;
(b) reacting, in the at least one reaction zone, a reaction mixture comprising an alkanoic acid and an alkylenating agent under conditions effective to form a crude acrylate product;
(c) regenerating, in the at least one regeneration zone, the respective catalyst; and
(d) separating at least a portion of the crude acrylate product to form a purified acrylate product;
wherein a ratio of the combined size of the at least one reaction zone to the combined size of the at least one regeneration zone ranges between 1:1 to 6:1.

2. The process of claim 1, further comprising the step of:
monitoring catalyst activity of the catalysts in the at least one reaction zone.

3. The process of claim 2, further comprising the step of:
discontinuing the reacting in at least one of the at least one reaction zone, based on the monitored catalyst activity.

4. The process of claim 3, wherein the discontinuing comprises discontinuing the reacting when acetic acid yield is less than 30%.

5. The process of claim 3, further comprising the step of:
regenerating the catalyst in the at least one discontinued reaction zone.

6. The process of claim 2, further comprising the step of:
discontinuing the regenerating in at least one of the at least one regeneration zone, based on the monitored catalyst activity.

7. The process of claim 6, further comprising the step of:
reacting, in the at least one discontinued regeneration zone, a reaction mixture comprising an alkanoic acid and an alkylenating agent over the respective catalyst and under conditions effective to form a crude acrylate product.

8. The process of claim 1, wherein the regenerating comprises the step of
feeding a regeneration stream to the at least one regeneration zone.

9. The process of claim 8, wherein the regeneration stream comprises at least one compound selected from the group consisting of oxygen, nitrogen, air, other inerts, and combinations thereof.

10. The process of claim 1, comprising at least two reaction zones.

11. The process of claim 10, wherein the at least two reaction zones are configured in parallel.

12. The process of claim 1, wherein the sizes of the at least one reaction zone and/or the at least one regeneration zone range from 5 m³ to 100 m³.

13. The process of claim 12, wherein the sizes of each zone are essentially the same.

14. The process of claim 1, wherein the catalysts in each zone are essentially the same.

15. A process for producing an acrylate product, the process comprising the steps of:
(a) providing a reaction system comprising three zones, each zone comprising a respective catalyst;
(b) reacting, in two of the three zones, a reaction mixture comprising an alkanoic acid and an alkylenating agent over the respective catalyst and under conditions effective to form a crude acrylate product;
(c) regenerating, in the other of the three zones, the respective catalyst; and
(d) separating at least a portion of the crude acrylate product to form a purified acrylate product.

16. The process of claim 15, wherein the reaction system comprises no more than three zones.

17. A process for producing an acrylate product, the process comprising the steps of:
(a) providing a reaction system comprising four zones, each zone comprising a respective catalyst;
(b) reacting, in three of the four zones, a reaction mixture comprising an alkanoic acid and an alkylenating agent over the respective catalyst and under conditions effective to form a crude acrylate product;
(c) regenerating, in the other of the four zones, the respective catalyst; and
(d) separating at least a portion of the crude acrylate product to form a purified acrylate product.

18. The process of claim 17, wherein the reaction system comprises no more than four zones.

19. A process for producing an acrylate product, the process comprising the steps of:
(a) providing a reaction system comprising five zones, each zone comprising a respective catalyst;
(b) reacting, in four of the five zones, a reaction mixture comprising an alkanoic acid and an alkylenating agent over the respective catalyst and under conditions effective to form a crude acrylate product;
(c) regenerating, in the other of the five zones, the respective catalyst; and
(d) separating at least a portion of the crude acrylate product to form a purified acrylate product.

20. The process of claim 19, wherein the reaction system comprises no more than five zones.

21. A process for producing an acrylate product, the process comprising the steps of:
(a) providing a reaction system comprising six zones, each zone comprising a respective catalyst;
(b) reacting, in five of the six zones, a reaction mixture comprising an alkanoic acid and an alkylenating agent over the respective catalyst and under conditions effective to form a crude acrylate product;
(c) regenerating, in the other of the six zones, the respective catalyst; and
(d) separating at least a portion of the crude acrylate product to form a purified acrylate product.

22. The process of claim 21, wherein the reaction system comprises no more than six zones.

23. A system comprising
at least one reaction zone, each reaction zone having a size and comprising a respective catalyst; and
at least one regeneration zone, each regeneration zone having a size and comprising a respective catalyst;
wherein a ratio of the combined size of the at least one reaction zone to the combined size of the at least one regeneration zone ranges between 1:1 to 6:1.

24. A process for producing an acrylate product, the process comprising the steps of:
(a) providing a reaction system comprising from two to six zones, each zone comprising a respective catalyst;
(b) reacting, in at least one of the zones, a reaction mixture comprising an alkanoic acid and an alkylenating agent over the respective catalyst and under conditions effective to form a crude acrylate product;
(c) regenerating, in at least one of the other zones, the respective catalyst; and
(d) separating at least a portion of the crude acrylate product to form a purified acrylate product.

25. The process of claim 24, wherein a rate of catalyst regeneration in the combined regeneration zones is substantially similar to a rate of deactivation in the combined reaction zones.

26. The process of claim 24, wherein step (c) comprises the step of contacting the respective catalyst with a regeneration feed to yield a regenerated catalyst.

27. The process of claim 26, wherein the regeneration feed comprises a compound selected from the group consisting of oxygen, nitrogen, air, carbon dioxide, inerts, or combinations thereof.
